SEQUENCE LISTING (1 sequence)

SEQ ID NO: 1 - protein, 7 aa, Homo sapiens
TFEAIRE

(19) United States
(12) Patent Application Publication
(10) Pub. No.: US 2023/0303979 A1
(43) Pub. Date: Sep. 28, 2023

(54) CELL HAVING GENE CORRECTED EX VIVO AND USE THEREOF

(71) Applicant: IUCF-HYU (INDUSTRY-UNIVERSITY COOPERATION FOUNDATION HANYANG, Seoul (KR)

(72) Inventors: Dong Ho CHOI, Seoul (KR); Sang Su BAE, Seoul (KR); Sung Ah HONG, Seoul (KR); Jae Min JEONG, Yangju-si (KR); Yo Han KIM, Seoul (KR)

(73) Assignee: IUCF-HYU (INDUSTRY-UNIVERSITY COOPERATION FOUNDATION HANYANG, Seoul (KR)

(21) Appl. No.: 18/021,135

(22) PCT Filed: Aug. 13, 2021
(86) PCT No.: PCT/KR2021/010828

§ 371 Date: Feb. 13, 2023

(30) Foreign Application Priority Data
Aug. 14, 2020 (KR) 10-2020-0102636

(51) Int. Cl. C12N 5/071; A61K 35/407; A61P 1/16

(52) U.S. Cl. CPC C12N 5/0672; A61K 35/407; A61P 1/16; C12N 2501/12; C12N 2510/00

(57) ABSTRACT

The present invention relates to a method for producing a cell having a genetic defect corrected, and a cell therapy agent comprising the cell, and, more particularly, to a method for producing a cell and a cell therapy agent comprising the cell, which comprise a method for isolating a cell from an individual, producing a chemically derived progenitor cell by processing a compound, and then correcting a mutant gene ex vivo.

The cell therapy agent of the present invention has significantly less side effects such as an off-target effect and tumor generation, and has shown a Tyrosinemia type I treatment effect that is more significant than when a simple cell is transplanted, and thus, the cell therapy agent is expected to be widely usable in treatment fields for diseases caused by gene mutation, including Tyrosinemia type I.

### TECHNICAL FIELD

The present invention relates to a method of preparing cells in which a genetic defect has been corrected and a cell therapeutic agent including the same, and more particularly, to a method of preparing cells which includes isolating cells from a subject, treating the cells with a compound to prepare chemical-derived progenitor cells, and correcting a mutant gene ex vivo, and a cell therapeutic agent including the same.

### BACKGROUND ART

Genetic mutations are caused by structural changes in DNA constituting genes in the process in which gene replication and division occur while cells divide. The causes of genetic mutations are diverse, such as advanced maternal age pregnancy, radiation, smoking, anticancer agents, toxic chemicals, and heavy metals, and there are thousands of diseases caused by a single gene defect. For example, the most common diseases are hemophilia, cystic fibrosis, sickle cell anemia, and thalassemia. Genetic mutations occur in about 1 in 100 persons. While some genetic abnormalities can be noticed at birth or within months, diseases, such as Huntington's disease, are caused by a single gene and developed later in adulthood.

Tyrosinemia type I (TH1), which is one of the rare diseases, is one of the autosomal recessive diseases caused by the lack of fumaryl acetoacetase (FAH), and it is known that this disease causes liver failure due to accumulation of toxic metabolites derived from the tyrosine metabolic pathway and can lead to hepatocellular carcinoma (HCC).

Currently, to treat tyrosinemia, 2-[2-nitro-4-trifluoromethylbenzoyl]-1,3-cyclohexane-dione (NTBC) is used, but this is not a radical treatment method. Some patients lack NTBC sensitivity, and there is still a risk of developing hepatocellular carcinoma during therapy.

In order to overcome the above problems of existing treatments, adenine base editors (ABEs) are administered through hydrodynamic tail vein injection using a non-viral delivery system to successfully correct the Fah gene mutation (Nature Biomedical Engineering volume 4, pages 125-130 (2020)). However, the in vivo treatment strategy cannot control, the CRISPR-mediated gene correction effect acting on non-target cells, and thus a conventional treatment strategy through gene correction has its limits.

### DISCLOSURE

### Technical Problem

The present invention is directed to providing a method of preparing cells in which a mutant gene has been corrected, which comprising:
(a) isolating cells from a subject;
(b) treating the isolated cells with a compound to prepare chemically-derived progenitor cells; and
(c) correcting a mutant gene of the chemically-derived progenitor cells ex vivo.

In addition, the present invention is directed to providing a cell therapeutic agent, which includes cells in which a mutant gene prepared by the above method has been corrected or a cell population thereof as an active ingredient.

In addition, the present invention is directed to providing a pharmaceutical composition for preventing or treating a genetic mutation-related disease, which includes the cell therapeutic agent.

However, technical problems to be solved in the present invention are not limited to the above-described problems, and other problems which are not described herein will be fully understood by those of ordinary skill in the art from the following description.

### Technical Solution

To achieve the purpose of the present invention described above, the present invention provides a method of preparing cells in which a mutant gene has been corrected, which comprising:
(a) isolating cells from a subject;
(b) treating the isolated cells with a compound to prepare chemically-derived progenitor cells; and
(c) correcting a mutant gene of the chemically-derived progenitor cells ex vivo.

In one embodiment of the present invention, the correcting of a gene may be correcting a gene by an adenine base editor or prime editing.

In another embodiment of the present invention, the corrected gene may be selected from the group consisting of fumarylacetoacetate hydrolase (Fah), ATPase copper transporting beta (ATP7B), Serpin family A member 1 (SERPINA1), ATP binding cassette subfamily B member 4 (ABCB4), aldolase, fructose-bisphosphate B (ALDOB), glycogen branching enzyme (GBE), Solute Carrier Family 25 Member 13 (SLC25A13), cystic fibrosis transmembrane conductance (CFTR), and ALMS1 Centrosome And Basal Body Associated Protein (ALMS1).

In still another embodiment of the present invention, the isolated cells may be primary hepatocytes.

In yet another embodiment of the present invention, a compound for treating the isolated cells may be one or more selected from the group consisting of a hepatic growth factor, A83-01, and CHIR99021.

In yet another embodiment of the present invention, the chemically-derived progenitor cells may be chemically-derived hepatic progenitor cells.

In addition, the present invention provides a cell therapeutic agent, which includes cells in which a mutant gene has been corrected, prepared by the above method, or a cell population thereof as an active ingredient.

In one embodiment of the present invention, the cell therapeutic agent may treat a disease caused by a genetic mutation.

In addition, the present invention provides a pharmaceutical composition for preventing or treating a genetic mutation-related disease, which includes the cell therapeutic agent.

In one embodiment of the present invention, the genetic mutation-related disease may be selected from the group consisting of tyrosinemia type I, phenylketonuria, Wilson's disease, alpha-1 antitrypsin deficiency, progressive familial intrahepatic cholestasis type 3, hereditary fructose intolerance, glycogen storage disease type IV, argininosuccinate lyase deficiency, citrin deficiency, neonatal intrahepatic cholestasis by citrin deficiency, cholesteryl ester storage disease, cystic fibrosis, hereditary hemochromatosis, and Alström syndrome.

### Advantageous Effects

It was confirmed that, when a cell therapeutic agent including cells in which a mutant gene has been corrected according to the present invention is used, compared to the case of transplanting conventional primary hepatocytes, there were fewer side effects such as an off-target effect and tumorigenesis, and a significant level of therapeutic effect on tyrosinemia type I was shown, so it can be effectively used for treatment of a disease caused by a genetic mutation, such as tyrosinemia type I.

However, it should be understood that the effect of the present invention is not limited to the above-described effects, and includes all effects that can be deduced from the configuration of the present invention described in the detailed description or claims of the present invention.

### DESCRIPTION OF DRAWINGS

FIG. 1A is a schematic diagram illustrating a method of preparing chemically-induced hepatic progenitor cells (HT1-mCdHs) by isolating hepatocytes from a HT1 mouse.

FIG. 1B is a result of immunofluorescence staining for isolated primary hepatocytes.

FIG. 1C is a result of confirming expression levels of gene markers by performing RT-qPCR on HT1-CdHs.

FIG. 1D is a result of immunofluorescence staining for HT1-CdHs.

FIG. 1E shows the expression profiles of general genes and cell cycle-related genes.

FIG. 1F shows a GSEA result which confirms a cell cycle and a stem module-specific gene set in HT1-CdHs.

FIG. 1G is a result of clustering analysis for HT1-CdHs.

FIG. 1H is a result of measuring doubling time after subculture for 3 passages of WT-mCdHs and HT1-mCdHs for 72 hours.

FIG. 1I is a result of confirming bright-field images at the early stage (p1) and the late stage (p21) of subculture of HT1-mCdHs.

FIG. 1J is a result of confirming bright-field images while culturing isolated primary hepatocytes in YAC and HAC media.

FIG. 1K is a result of confirming whether a HT1-mCdHs-related gene marker is expressed by performing RT-qPCR on HT1-mCdHs.

FIG. 2 is a result of confirming the characteristics of HT1-mCdHs by bright-field, ICG uptake level, PAS staining, and immunofluorescence staining under the condition of hepatocyte differentiation.

FIG. 3A is a schematic diagram illustrating a method of correcting a gene causing HT1.

FIG. 3B is a schematic diagram illustrating the structures of plasmids encoding ABEmax, NG-ABEmax and NG-ABE8e.

FIG. 3C shows the structures of pegRNA1 and sgRNA1b used in prime editing technology.

FIG. 3D shows a heatmap by visualizing an A-to-G conversion rate through high-throughput sequencing, after correction of a gene of HT-mCdHs using ABE technology and PE technology.

FIG. 3E specifically shows an A-to-G conversion rate according to a base position in HT-mCdHs that have been gene-corrected using ABE technology.

FIG. 3F is a result of confirming an insertion-and-deletion (indel) ratio in HT-mCdHs that have been gene-corrected using ABE technology FIG. 3G is a result showing target sites of pegRNA and nicking sgRNA, respectively, and FIG. 3H is a result specifically showing the sequences of the target sites.

FIG. 3I the structure of pegRNA1 designed to correct a mutation causing a disease.

FIG. 3J is a result of confirming an indel ratio after transformation of HT1-mCdHs with pegRNAs having prime-binding sites with different lengths (n=1˜4).

FIG. 4A is a schematic diagram illustrating a process of screening Fah gene-corrected cells from ABE-treated mCdHs.

FIG. 4B is a result of confirming base change levels through high-throughput sequencing on bulk cells after selecting gene-corrected cells (HT1-mCdHs-ABE #1, HT1-mCdHs-ABE #2) from ABE-treated mCdHs.

FIG. 4C is a result of confirming the off-target effect of HT1-mCdHs-ABE #1-1 using Cas-OFFinder.

FIG. 5A is a schematic diagram illustrating the process of transplanting ABE-treated HT1-mCdHs into a HT1 mouse.

FIG. 5B is a Kaplan-Meier survival curve of HT1 mice according to the presence or absence of ABE-treated HT1-mCdHs transplantation.

FIG. 5C is a result of confirming expression levels of aspartate transaminase (AST), alanine transaminase (ALT), total bilirubin and albumin (ALB) in sera of HT1-mCdHs, HT1-mCdHs-ABE #1, HT1-mCdHs-ABE #2, HT1-mCdHs-ABE #1-1 and WT-mPH.

FIG. 5D is a result of confirming a therapeutic effect by immune cell staining for Fah gene in the liver 40, 130 and 180 days after transplantation of HT1-mCdHs-ABE #1-1 into HT1 mice.

FIG. 5E is a result of confirming a therapeutic effect by immune cell staining for Fah gene in the liver of the WT-mPHs-transplanted HT1 mouse.

FIG. 5F is a result of confirming whether a mature hepatocyte-specific marker is expressed through RT-qPCR, after transplantation of HT1-mCdHs-ABE #1-1 into a mouse and then reisolation.

FIG. 5G is a result of confirming an edited nucleotide rate 180 days after transplantation of HT1-mCdHs-ABE #1-1 into a HT1 mouse.

FIG. 5H is an image of the liver of a HT1-mCdHs-ABE #1-1 or WT-mPHs-transplanted HT1 mouse (the arrow indicates hepatocellular carcinoma).

FIG. 5I shows results of immunostaining for Fah gene and H&E staining for liver tissue 180 days after transplantation of HT1-mCdHs-ABE #1-1 into HT1 mice, and FIG. 5J shows results of immunostaining for Fah gene and H&E staining for liver tissue 130 days after transplantation of HT1-mCdHs-ABE #1-1.

FIG. 5K is a result of immunohistochemical staining for AFP in liver tissue of a HT1 mouse 130 days after transplantation of HT1-mCdHs-ABE #1-1.

FIG. 5L is a result of confirming the percentage of each nucleotide by high-throughput sequencing in hepatocellular carcinoma cells indicated by the arrow in FIG. 5H.

FIG. 6A is a schematic diagram illustrating a method of transplanting HT1-mCdHs-PE3b in which a gene has been corrected by PE into a HT1 mouse.

FIG. 6B is a result of confirming a Kaplan-Meier survival curve in mice (n=13) transplanted with cells in which a gene has been corrected by PED or control mice (n=9) into which only PBS is injected.

FIG. 6C is a result of confirming expression levels of aspartate transaminase (AST), alanine transaminase (ALT), total bilirubin and albumin (ALB) in sera of HT1-mCdHs-PE3b- and WT-mPHs-transplanted mice.

FIG. 6D is a result of immunohistochemical staining of Fah gene 80 or 140 days after transplantation of HT1-mCdHs-PE3b into a HT1 mouse.

FIG. 6E is a result of confirming an edited nucleotide rate 140 days after transplantation of HT1-mCdHs-PE3b into a HT1 mouse.

### MODES OF THE INVENTION

The present inventors confirmed that, when using a cell therapeutic agent including cells in which a mutant gene has been corrected according to the present invention, compared to conventional primary hepatocyte transplantation, there are fewer side effects such as an off-target effect and tumorigenesis, and a significant level of therapeutic effect on tyrosinemia type I is shown, and thus the present invention was completed.

Therefore, as an aspect of the present invention, the present invention provides a method of preparing mutant gene-corrected cells, which comprises:
(a) isolating cells from a subject;
(b) treating the isolated cells with a compound to prepare chemically-derived progenitor cells; and
(c) correcting a mutant gene of the chemically-derived progenitor cells ex vivo.

In the present invention, the correcting of a gene may be correcting a gene by an adenine base editor or prime editing.

The term “adenine base editor (ABE)” used herein may be constructed by fusing any natural deaminase (ecTadA) and adenine deaminase variant (ecTadA*) with the N-terminus of Cas9 nickase to correct adenine to guanine, and types of ABEs may include, but are not limited to, ABE6.3 ABE7.8, ABE7.9, ABE 7.10, NG-ABEmax, NG-ABE8e and ABEmax depending on the version. The “composition for cytosine (C) base editing, which includes adenine deaminase and CRISPR associated protein 9 (Cas9) protein or a functional analogue thereof” may be referred to as “ABEs.”

The “adenine deaminase” according to the present invention is an enzyme involved in the removal of an amino acid from adenine and production of hypoxanthine, and although this enzyme is rarely found in higher animals, it is reported that the enzyme is present in small amounts in the muscles and milk of cows, or the blood of rats, and present in large amounts in the intestines of crayfish or insects. Adenine deaminase includes natural adenine deaminase such as ecTadA, but the present invention is not limited thereto. Adenine deaminase includes a variant of adenine deaminase such as a mutant of ecTadA (ecTadA*), but the present invention is not limited thereto.

The “CRISPR-associated protein 9 (Cas9)” is a protein playing a pivotal role in the immunological defense of certain bacteria against DNA viruses, and is widely used in genetic engineering applications. Since the major function of the protein is to cleave DNA, the protein can be applied to change a cell's genome. Technically, Cas9 is an RNA-guided DNA endonuclease associated with the Clustered Regularly Interspaced Short Palindromic Repeats (CRISPR) adaptive immune system in Streptococcus pyogenes, and Cas9 works in the mechanism that unwinds foreign DNA strands, identifies a site complementary to the 20-nucleotide spacer region of guide RNA, and considers the DNA as invading DNA when the DNA is complementary to the guide RNA, and cuts it.

The “prime editing technology” used herein is fourth generation gene scissor technology developed to improve the low precision of CRISPR gene scissor technology, and unlike conventional CRISPR technology, this technology is characterized by cutting only one strand of the two strands of target DNA, and comprises a fusion protein including nicking sgRNA and prime editing guide RNA (pegRNA), in which the pegRNA consists of an RNA spacer, a reverse transcription template (RTT) and a primer-binding site, and the composition used for prime editing may be PE or PE3, but the present invention is not limited thereto.

In the present invention, the gene correction may be performed by the electroporation treatment of target cells with a composition for prime editing or an ABE composition, but the present invention is not limited thereto.

In the present invention, the isolated cells may be primary hepatocytes, and a gene corrected by ABE or PE is a gene causing a disease through a mutation. The gene may be, but is not limited to, fumarylacetoacetate hydrolase (Fah), ATPase copper transporting beta (ATP7B), Serpin family A member 1 (SERPINA1), ATP binding cassette subfamily B member 4 (ABCB4), aldolase, fructose-bisphosphate B (ALDOB), glycogen branching enzyme (GBE), Solute Carrier Family 25 Member 13 (SLC25A13), cystic fibrosis transmembrane conductance (CFTR) or ALMS1 Centrosome And Basal Body Associated Protein (ALMS1) genes, and preferably, the fumarylacetoacetate hydrolase (Fah) gene.

The isolated cells may be prepared as chemically-derived progenitor cells having stem cell-like ability through chemical treatment, and more particularly, chemically-derived hepatic progenitor cells (CdHs). The CdHs may be prepared through reprogramming of human adult hepatocytes by the composition of a reprogramming medium for reprogramming into hepatic progenitor cells, which includes one or more selected from the group consisting of a hepatic growth factor (HGF), a TGF-β inhibitor (A83-01) and a GSK-3 inhibitor (CHIR99021). The CdHs of the present invention may express genes from the epithelial lineages of the liver and bile duct, may be stained with a hepatic progenitor cell-specific marker, and may differentiate into cholangiocytes and hepatocytes, and thereby have the characteristics of bipotent hepatic stem cells.

The present inventors confirmed, through specific experiments, that, when cells of the present invention in which a gene has been corrected ex vivo are used, a disease caused by a genetic mutation including tyrosinemia may be significantly treated.

In one embodiment of the present invention, it was confirmed that cells in which a gene had been corrected by the gene editing technology of the present invention did not exhibit an off-target effect in parts other than a target gene, so there was no unexpected side effect (see Example 4).

In another embodiment of the present invention, even when NTBC was completely withdrawn from the drinking water of a HT1 mutant mouse model, in the case of a control mouse and a mouse with simple transplantation of HT1-mCdHs, on day 90, it was confirmed that both died. However, in the case of the cells of the present invention in which a gene has been corrected ex vivo by ABE, it was confirmed that two of 9 mice survived for 180 days or longer, indicating that the cell therapeutic agent of the present invention can treat a mutation-related disease at a significant level (see Example 5-1), and confirming that the cells of the present invention are not related with the occurrence of hepatocellular carcinoma (HCC) (see Example 5-2). In addition, it was confirmed that 7 of 13 mice transplanted with HT1-mCdHs-PE3b corrected by PE, not ABE, survived for 160 days or longer (see Example 6).

From the results of the specific experiments described above, the present inventors confirmed that, when using the cells of the present invention and a cell therapeutic agent including the same, diseases caused by genetic mutations can be treated without side effects.

As another aspect of the present invention, the present invention provides a cell therapeutic agent, which includes mutant gene-corrected cells prepared by the above method of the present invention or a cell population thereof as an active ingredient.

As still another aspect of the present invention, the present invention provides a pharmaceutical composition for preventing or treating a genetic mutation-related disease, which includes the cell therapeutic agent.

The term “prevention” used herein refers to all actions of inhibiting a disease caused by a genetic mutation or delaying the occurrence of the disease by administration of the pharmaceutical composition according to the present invention.

The term “treatment” used herein refers to all actions involved in alleviating or beneficially changing symptoms of a disease caused by a genetic mutation by administration of the pharmaceutical composition according to the present invention.

The pharmaceutical composition according to the present invention may include a cell therapeutic agent including the gene-corrected cells of the present invention as an active ingredient, and may further include a pharmaceutically acceptable carrier. The pharmaceutically acceptable carrier is generally used in formulation, and may be, but is not limited to, saline, distilled water, Ringer's solution, buffered saline, cyclodextrin, a dextrose solution, a maltodextrin solution, glycerol, ethanol, or a liposome. If needed, the pharmaceutically composition may further include other conventional additives including an antioxidant, a buffer and the like. In addition, by additionally adding a diluent, a dispersant, a surfactant, a binder or a lubricant, the pharmaceutical composition may be formulated as an injectable form such as an aqueous solution, an emulsion or a suspension, a pill, a capsule, a granule or a tablet. Suitable pharmaceutically acceptable carriers and their formulations may be properly formulated according to each ingredient using a method disclosed in the Remington's Pharmaceutical Science. The pharmaceutical composition of the present invention is not limited in dosage form, and thus may be formulated as an injection, an inhalant, or a dermal preparation for external use.

The pharmaceutical composition of the present invention may be orally or parenterally (e.g., intravenously, subcutaneously, intraperitoneally, or topically) administered by a desired method, and a dose may depend on a patient's condition and body weight, the severity of a disease, a dosage form, an administration route and duration, and may be appropriately selected by those of ordinary skill in the art.

The composition according to the present invention is administered at a pharmaceutically effective amount. In the present invention, the “pharmaceutically effective amount” used herein refers to an amount sufficient for treating a disease at a reasonable benefit/risk ratio applicable for medical treatment, and an effective dosage may be determined by parameters including the type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the pharmaceutical composition of the present invention may depend on a patient's age, sex, condition, body weight, absorbance of an active ingredient in the body, inactivation rate and excretion rate, disease type, or drugs used in combination, and generally, 0.001 to 150 mg, and preferably 0.01 to 100 mg per kg of body weight may be administered daily or every other day, or one to three times a day. However, the effective amount may be increased or decreased depending on the route of administration, the severity of obesity, sex, a body weight or age, and thus it does not limit the scope of the present invention in any way.

The present inventors investigated the use of a pharmaceutical composition including a cell therapeutic agent including the gene-corrected cells of the present invention for prevention and treatment of a genetic mutation-related disease through specific experimental examples.

In the present invention, the genetic mutation-related disease may be selected from the group consisting of tyrosinemia type I, phenylketonuria, Wilson's disease, alpha-1 antitrypsin deficiency, progressive familial intrahepatic cholestasis type 3, hereditary fructose intolerance, glycogen storage disease type IV, argininosuccinate lyase deficiency, citrin deficiency, neonatal intrahepatic cholestasis by citrin deficiency, cholesteryl ester storage disease, cystic fibrosis, hereditary hemochromatosis, and Alström syndrome.

In yet another aspect of the present invention, the present invention provides a method of preventing or treating a genetic mutation-related disease, which includes administering the pharmaceutical composition to a subject.

The term “subject” used herein refers to a subject in need of treatment of a disease, and more specifically, a mammal such as a human or a non-human primate, a mouse, a rat, a dog, a cat, a horse, or a cow.

In yet another aspect of the present invention, the present invention provides a use of the pharmaceutical composition for prevention or treatment of a genetic mutation-related disease.

Hereinafter, to help in understanding the present invention, exemplary examples will be suggested. However, the following examples are merely provided to more easily understand the present invention, and not to limit the present invention.

### EXAMPLES

### Example 1. Experiment Preparation and Experimental Methods

1-1. Preparation of Laboratory Animals and Disease Models

Tyrosinemia type I (HT1) mice used in the experiments were provided from Hyoungbum (Henry) Kim. Experiments were performed on 6- to 8-week-old male and female mice, and housed and cared under aseptic conditions in accordance with the Principles of Laboratory Animal Care and the Guide Regulations for the Use of Laboratory Animals of HYU Industry-University Cooperation Foundation (2018-0196A). Liver damage was induced in the HT1 mice by non-treatment of NTBC for 1 week.

1-2. Isolation and Cell Culture of Primary Hepatocytes

To isolate Fah^{-/-} mouse primary hepatocytes, livers of HT1 mice were perfused through the portal vein using solution A (0.19 g/L EDTA (Sigma-Aldrich), 8 g/L NaCl, 0.4 g/L KCl, 0.078 g/L NaH₂PO₄.2H₂O, 0.151 g/L Na₂HPO₄.12H₂O, and 0.19 g/L HEPES) at 37° C. for 5 minutes, and then perfused with solution B (0.3 g/L collagenase (Worthington Biochemical), 0.56 g/L CaCl₂, 8 g/L NaCl, 0.4 g/L KCl, 0.078 g/L NaH₂PO₄.2H₂O, 0.151 g/L Na₂HPO₄.12H₂O, and 0.19 g/L HEPES) at 37° C. for 8 minutes. Variable primary hepatocytes were obtained by isopycnic centrifugation in Percoll solution (GE Healthcare). Isolated Fah^{-/-} mouse primary hepatocytes were seeded in a collagen-coated plate at 2,000 cells/cm². Subsequently, the cells were cultured in William's E medium (Gibco) in a humidified atmosphere containing 5% CO₂ at 37° C.

To generate chemically-derived hepatic progenitor cells (hereinafter, HT1-mCdHs) from the HT1 primary hepatocytes, one day after seeding, the medium was replaced with a reprogramming medium [DMEM/F-12 medium containing 1% fetal bovine serum (FBS; Gibco), 1% insulin-transferrin-selenium (Gibco), 0.1 μM dexamethasone (Sigma-Aldrich), 10 mM nicotinamide (Sigma-Aldrich), 50 μM [3-mercaptoethanol (Sigma-Aldrich), 1% penicillin/streptomycin (Gibco), 20 ng/mL of an epidermal growth factor (Peprotech), 20 ng/mL of a hepatic growth factor (Peprotech), 4 μM A83-01 (Sigma-Aldrich) and 3 μM CHIR99021 (Sigma-Aldrich)]. The reprogramming medium was changed every 2 days. First, the cells were subcultured every 4 to 6 days after separating the cells from the plate using 1× TrypLE Express enzyme (Gibco), diluting the detached cells in a fresh medium in a ratio of 1:4, and plating the cells on a fresh collagen-coated plate. After base editing, the bulk population of the cells was diluted and seeded in a 96-well plate to select a single cell-derived clone.

To induce differentiation into hepatocytes, HT1-mCdHs were seeded in a collagen-coated plate at 1,000 cells/cm². After one day of culture, the medium was replaced with a differentiation medium consisting of a reprogramming medium supplemented with 20 ng/mL oncostatin M (Prospec) and 10 μM dexamethasone, and the medium was replaced every two days. After 6 days, the cells were covered with Matrigel (Corning) diluted in a differentiation medium in a ratio of 1:7 and cultured for 2 days or more.

To induce differentiation into cholangiocytes, HT1-mCdHs were harvested by treatment with 1× TrypLE Express enzyme, and resuspended in a 6-well plate at a density of DMEM/F-12 medium [referred to as a cholangiocyte differentiation medium (CDM)] containing 10% FBS and 20 ng/mL of a hepatic growth factor at a density of 1×10⁵ cells/well. The CDM was mixed on ice with an equal volume of collagen type I (pH 7.0), and incubated at 37° C. for 30 minutes for solidification. Subsequently, the cells were overlaid with the mixture and cultured for 7 days. The medium was replaced every 2 days.

To compare HT1-mCdHs with chemically-induced hepatic progenitors (CLiPs) manufactured by Katsuda et al. (Cell Stem Cell Volume 20, Issue 1, 5 Jan. 2017, Pages 41-55), isolated mouse primary hepatocytes (PHs) from HT1 mice incubated in YAC-containing medium for 7 days according to the method of Katsuda et al., and then harvested the cells to perform RT-qPCR analysis.

1-3. Immunostaining

For immunocytochemistry, the cells were fixed in 4% paraformaldehyde at 4° C. overnight, the fixed cells were washed with PBS and treated with PBS containing 0.2% Triton X-100 for 10 minutes at room temperature. Subsequently, the cells were treated with a blocking solution consisting of 1% bovine serum albumin, 22.52 ng/mL glycine and 0.1% Tween 20 in PBS at room temperature for 1 hour, and cultured with primary antibodies diluted in a blocking solution at 4° C. overnight. After washing, the primary antibodies were detected using Alexa Fluor 488-conjugated or Alexa Fluor 594-conjugated secondary antibodies (Thermo Fisher Scientific). Nuclei were counterstained with Hoechst 33342 (1:10,000, Molecular Probes). The primary antibodies used in this study are listed in the Key Resources Table. The stained cells were visualized under a TCS SP5 confocal microscope (Leica).

For immunohistochemistry, liver tissue samples were fixed in 10% formalin and embedded in paraffin. Sections were subjected to immunohistochemical staining. Immunohistochemical staining was performed using the Dako REAL™ EnVision™ Detection System (Dako). Anti-FAH antibodies (Yecuris, 20-0034) were used as primary antibodies, and nuclei were counterstained with hematoxylin. Stained tissue was observed under a virtual microscope Axio Scan.Z1 (Zelss).

1-4. RT-PCR Analysis

Total RNA was isolated using Trizol Reagent (Gibco), 1 μg of an RNA sample was reverse-transcribed using a Transcriptor First Strand cDNA synthesis kit (Roche). RT-PCR was performed using the CFX Connect Real-Time PCR Detection System (Bio-Rad). Each reaction solution contained 10 μL of qPCR PreMix (Dyne Bio) and 1 μL of cDNA, and oligonucleotide primers, and analyzed three times for each gene. The PCR cycle consisted of 40 cycles of 95° C. for 20 seconds and 60° C. for 40 seconds as one cycle. Melting curves and melting peak data were obtained to characterize the PCR products. The primer sequences are listed in Table 1 below.

TABLE 1

| | | |
|---|---|---|
| Gene | Primer | Sequence (5′ to 3′) |

Primers for qRT-PCR

| | | |
|---|---|---|
| Alb | Fwd | GGCTACAGCGGAGCAACTGA |
| | Rev | GCCTGAGAAGGTTGTGGTTGTG |
| Sox9 | Fwd | TCCTAACGCCATCTTCAAGG |
| | Rev | ACGTCTGTTTTGGGAGTGGT |
| Epcam | Fwd | TCGTGGTGGTGTTAGCAGTC |
| | Rev | TCTGTGTATCTCACCCATCTCC |
| Afp | Fwd | CGTCCCTCCACCATTCTCTG |
| | Rev | CGTGCTGCTCCTCTGTCATT |
| Krt19 | Fwd | TTCCGGACCAAGTTTGAGAC |
| | Rev | CCTCGTGGTTCTTCTTCAGG |
| Itga6 | Fwd | GGATCATCCTCCTGGCTGT |
| | Rev | TGTGGTAGGTGGCATCGTAA |
| Cd44 | Fwd | GGCTTATCATCTTGGCATCC |
| | Rev | CTGTTCCATTGCCACTGTTG |
| Cd90 | Fwd | AACTTCACCACCAAGGAT |
| | Rev | TTGTCTCTATACACACTGATACT |
| Hnf6 | Fwd | GACCATGGCCTGTGAAACTC |
| | Rev | TGAAACTACCGCTCACGTTG |
| Asgr1 | Fwd | CAGCTCTGTGAGGCCTTGGA |
| | Rev | GGGCCCGTTCTGGTCAGTTA |
| Hnf4a | Fwd | ATCGRCAAGCCTCCCTCTGC |
| | Rev | GACTGGTCCCTCGTGTCACATC |
| Cyp1a2 | Fwd | AGGAGCTGGACACGGTGGTT |
| | Rev | AGGTGTCCCTCGTTGTGCTG |
| Cyp2c9 | Fwd | TGACTTGTTTGGAGCTGGGACAGA |
| | Rev | ACAGCATCTGTGTAGGGCATGT |
| Aat | Fwd | AATGGAAGAAGCCATTCGAT |
| | Rev | AAGACTGTAACTGCTGCAGC |
| Ttr | Fwd | AGTCCTGGATGCTGTCCGAG |
| | Rev | TTCCTGAGCTGCTAACACGG |
| Arg1 | Fwd | ACAGCTAATGAGGACGACAG |
| | Rev | CCACCCAAATGACACATAGG |
| Cps1 | Fwd | TGAGACAGGCCAAAGAGATTGGGT |
| | Rev | TGCTCCTGGCCATTGTAGGTAACA |
| Fxr | Fwd | TGTGAGGGCTGCAAAGGTT |
| | Rev | ACATCCCCATCTTGGAC |
| Oct | Fwd | TCCTGCTCAACAAGGCAGCTCTTA |
| | Rev | TCACGGCCTTTCAGCTGTACTTGA |
| Cftr | Fwd | GGTCATAGAGCAGGGCAATG |
| | Rev | TGCACTTCTTCCTCCGTCTC |
| Ae2 | Fwd | GACTCCTTTCCCTGTGTGGA |
| | Rev | GAAGCATCCGCTCTTTCTTG |
| Aqpr1 | Fwd | CTGTGCGTTCTGGCTACCAC |
| | Rev | GCACAGCAGAGCCAAATGAC |
| Aqpr9 | Fwd | CTCAGTCCCAGGCTCTTCAC |
| | Rev | TAAGACCTCCCAGGAAAGCA |
| Grhl2 | Fwd | GTTCGATGCTCTGATGCTGA |
| | Rev | GCAGCCCGTACTTCTCAGAC |
| Gabdh | Fwd | CCAATGTGTCCGTCGTGGAT |
| | Rev | TTGCTGTTGAAGTCGCAGGAG |

Primers for high-throughput sequencing

| | | |
|---|---|---|
| Fah | 1^{st}_Fwd | AGTAATGCCAGGTCCTCAGG |
| | 1^{st}_Rev | GTCAGCTCCATCCTTCCACT |
| | 2^{nd}_Fwd | ACACTCTTTCCCTACACGAC |
| | | GCTCTTCCGATCTCTCCATG |
| | | GCAGGCTTTCTTC |
| | 2^{nd}_Rev | GTGACTGGAGTTCAGACGTG |
| | | TGCTCTTCCGATCTCCACAC |
| | | CCACAGAGTCAGAA |

1-5. Library Preparation and Transcriptome Sequencing

Total RNA concentration were calculated using Quant-IT RiboGreen (Invitrogen, USA), and integrity values were accessed with TapeStation RNA ScreenTape (Agilent Technologies, USA). Only high-quality RNA confirmed to have an integrity value of more than 7.0 was selected and used for library construction, and 1 mg of a total RNA library for each sample was independently prepared using the Illumina TruSeq Stranded mRNA Sample Prep Kit (Illumina, Inc., San Diego, CA, USA).

In the initial stage of library preparation, mRNA molecules including poly-A was purified with poly-A attached magnetic beads, and the purified mRNA was fragmented using bivalent cations at elevated temperatures. The cut mRNA fragment was copied into the first strand of cDNA using SuperScript II reverse transcriptase (Invitrogen), random primers and DNA polymerase I, and the complementary strand of cDNA was synthesized using DNA polymerase I, RNase H and dUTP.

A single ‘A’ base was added to the cDNA fragment obtained through the above steps, and an adapter was attached to perform final repairing, thereby finally forming a cDNA library. Libraries were quantified using a KAPA library quantification kit for the Illumina sequencing platform in accordance with a qPCR quantification protocol guide (Kapa Biosystems, USA), and verified with TapeStation D1000 ScreenTape (Agilent Technologies). The indexed library was paired-end sequenced with Illumina HiSeq 2500 (Illumina, Inc.) at Macrogen, Inc.

1-6. Bioinformatic Analysis

The standard Illumina pipeline and real-time analysis tools were used to generate FASTQ data from raw image processing, base calling and paired-end RNA sequencing data. 100 bp×2 read sequences were pre-processed using Sickle (V1.33, to trim low-quality sub sequences, and aligned to the hg19 human reference genome using RSEM (v1.2.31) and STAR (v2.5.2b).

Clustering analysis was performed using Cluster3.0 (http://eisenlab.org/) and a heatmap (v3.3.2, https://www.r-project.org). Gene set enrichment analysis (GSEA) scores were generated for the gene sets in the C5 and C8 bp data sets, and normalized enrichment scores and p-values were calculated using GSEA software (https://www.gsea-msigdb.org/gsea/index.jsp).

1-7. Calculation of Doubling Time

HT1-mCdHs were seeded at a density of 1×10⁴ cells/well on a collagen-coated 6-well plate, and cell numbers were counted on day 3 and 7. The doubling time was calculated using the following formula as described at http://www.doubling-time.com/compute.php:

Doubling time=time*log(2)/[log(final concentration)−log(initial concentration)] [Formula]

### 1-8. ICG Uptake Detected by PAS Staining

To detect glycogen, as recommended by a supplier, cells were stained with PAS reagent using a PAS staining kit (Abcam) in the presence or absence of diastase (Sigma). To detect ICG (Sigma) uptake, the cells were incubated in a medium containing 1 mg/mL of ICG at 37° C. for 30 minutes, and examined under a phase-contrast microscope.

1-9. Construction of sgRNA and pegRNA Expression Plasmids

To construct a sgRNA expression plasmid, complementary oligos representing the target sequence were annealed and cloned into pRG2 (Addgene #104174). To construct a pegRNA expression plasmid, complementary oligos representing the target sequence, a sgRNA scaffold and a 3′ extension was annealed and cloned into the pU6-pegRNA-GG-acceptor (Addgene #132777).

1-10. Transfection of HT1-mCdHs

Transfection was performed by electroporation using the Amaxa 4-D device (Lonza) or Neon Transfection System (Thermo Fisher). For the Amaxa 4-D device, the P3 Primary Cell 4D-Nucleofector X Kit (program EX-147) was used. 200,000 HT1-mCdHs were electroporated with 750 ng of ABEmax-encoding plasmid (Addgene, #112095) and 250 ng of sgRNA-encoding plasmid. Using the Neon Transfection System, 100,000 HT1-mCdHs were transfected with 900 ng of PE2-encoding plasmid (Addgene #132775); 300 ng of pegRNA-encoding plasmid and 83 ng of nicking guide RNA (ngRNA)-encoding plasmid; or 900 ng of NG-ABE-encoding plasmid (NG-ABE8e, Addgene #138491) and 250 ng of sgRNA-encoding plasmid by electroporation according to the following parameters (voltage: 1,200V duration: 50 ms, and number: 1). The NG-ABEmax-encoding plasmid was formed in the laboratory of the present inventors based on an appropriate backbone plasmid (Addgene #112095).

The transfected cells were cultured in a reprogramming medium for 3 days, treated with TrypLE Express Enzyme, and centrifuged, frozen and prepared for high-throughput sequencing. For freezing, the cells were resuspended in a reprogramming medium and then stored at −80° C.

1-11. High-Throughput Sequencing

For high-throughput sequencing, cell pellets were resuspended in 100 μL of proteinase K extraction buffer [40 mM Tris-HCl (pH 8.0, Sigma), 1% Tween-20 (Sigma), 0.2 mM EDTA (Sigma), 10 mg of proteinase K, 0.2% Nonidet P-40 (VWR Life Science)], cultured at 60° C. for 15 minutes, and then heated at 98° C. for 5 minutes.

An ABE target site was amplified from extracted genomic DNA using SUN-PCR blend (Sun Genetics). PCR products were purified using Expin™ PCR SV mini (GeneAll) and sequenced using the MiniSeq Sequencing System (Illumina). The results were analyzed using Cas-Analyzer (http://www.rgenome.net/cas-analyzer/), BE-analyzer (BE-Analyzer; http://www.rgenome.net/be-analyzer/), and primers (the used primers are shown in Table 1).

1-12. Restriction Enzyme V-Coupled Digested Genome Sequencing (Digenome-Seq)

Genomic DNA was extracted from HT1-mCdHs using the DNeasy Blood & Tissue Kit (Qiagen). 8 μg of the genomic DNA was incubated with 32 μg of ABE pre-incubated with 24 μg of in vitro-transcribed sgRNA at room temperature for 5 minutes, and then 300 μL of 2×BF buffer (Biosesang) was added, followed by adjusting the reaction volume to 600 μL. The mixture was incubated at 37° C. for 16 hours. After RNase A (50 μg/mL, Thermo Scientific) treatment at 37° C. for 15 minutes, ABE-treated genomic DNA was purified using the DNeasy Blood & Tissue Kit. 3 μg of the purified DNA was digested with 8 units of Endonuclease V (New England Biolabs) in a 200 μL reaction solution at 37° C. for 2 hours. The genomic DNA was then purified using the DNeasy Blood & Tissue Kit. Whole genome sequencing was performed with 1 μg of the digested DNA using the HiSeq X Ten Sequencer (Illumina) at Macrogen.

1-13. In Vitro Transcription of sgRNA

To generate a template for in vitro transcription, forward oligos containing a T7 RNA polymerase promoter and a target sequence and reverse oligos containing a guide RNA scaffold were purchased from Macrogen, and extended using Phusion DNA Polymerase (Thermo Scientific). The extended DNA was extended using Expin PCR SV mini (GeneAll), and transcribed with T7 RNA Polymerase (New England Biolabs). After incubation at 37° C. for 16 hours, a DNA template was digested with DNase I (New England Biolabs), and an RNA product was purified with Expin PCR SV mini (GeneAll).

1-14. Cell Transplantation

To transplant cells that had been subjected to ex vivo gene manipulation into mice, 7 days before cell transplantation into mice, NTBC was withdrawn from drinking water. 1×10⁶ cells in 100 μL PBS were transplanted into the inferior pole of the spleen. NTBC was temporarily provided every 3 days when the mice reached 80% of their initial weight, and completely withdrawn from drinking water after 90 days in HT1-mCdHs-ABE-transplanted mice, and after 60 days in HT1-mCdHs-PE3b-transplanted mice. After transplantation, serum was collected for biomarker analysis. Serum was diluted at a ratio of 1:4 to obtain an average.

1-15. Ploidy Analysis

For ploidy analysis, HT1 mPHs, mCdHs and mCdHs-ABE #1-1 cells were separated from plates by trypsinization and then incubated with 15 μg/mL of Hoechst 33342 and 5 μM reserpine at 37° C. for 30 minutes. The incubated cells were used to analyze cell ploidy using FACSCanto II (BD Biosciences) as described in Duncan et al. (Nature volume 467, pages 707-710 (2010)).

1-16. Statistical Analysis

Doubling time experiments and qRT-PCR were performed with triplicate biological replicates. Quantitative data is presented as means±standard deviations (SD) with inferential statistics (p-values). Survival levels were analyzed by Kaplan-Meier curve using GraphPad Prism 7 (GraphPad). Statistical significance was evaluated by two-tailed t-tests set at *p<0.05, **p<0.01, and ***p<0.001.

### Example 2. Preparation and Characterization of Chemically-Derived Hepatic Progenitor Cells (mCdHs) Derived from HT1 Model Mouse

2-1. Preparation of HT1-mCdHs

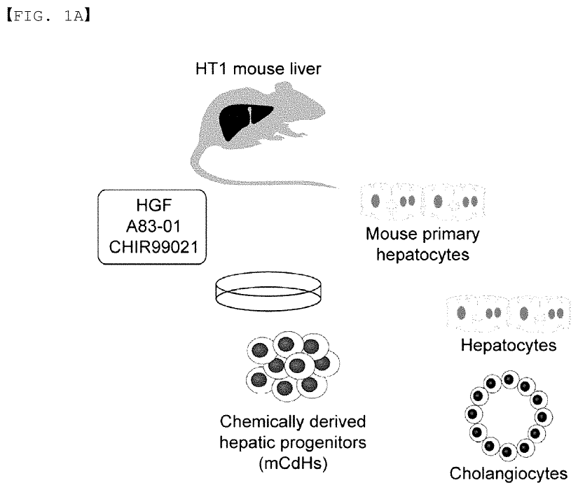

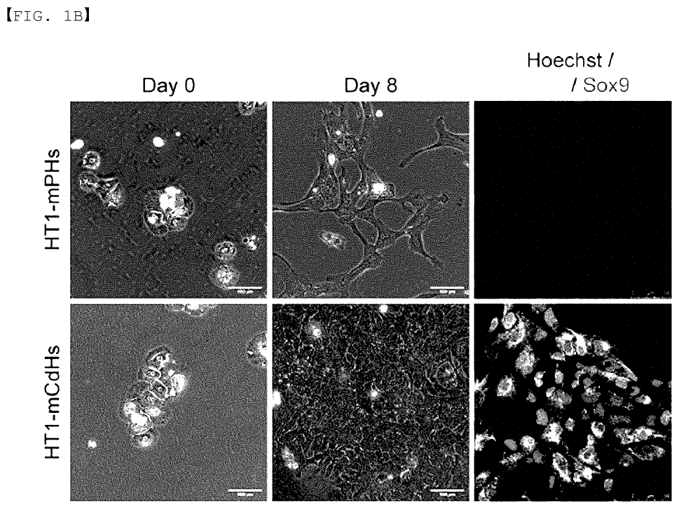

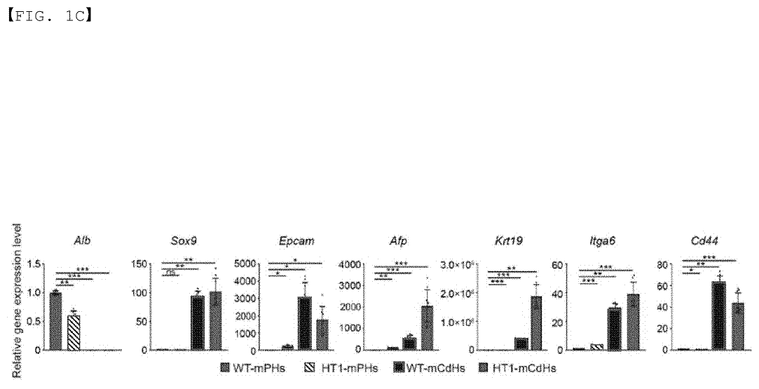

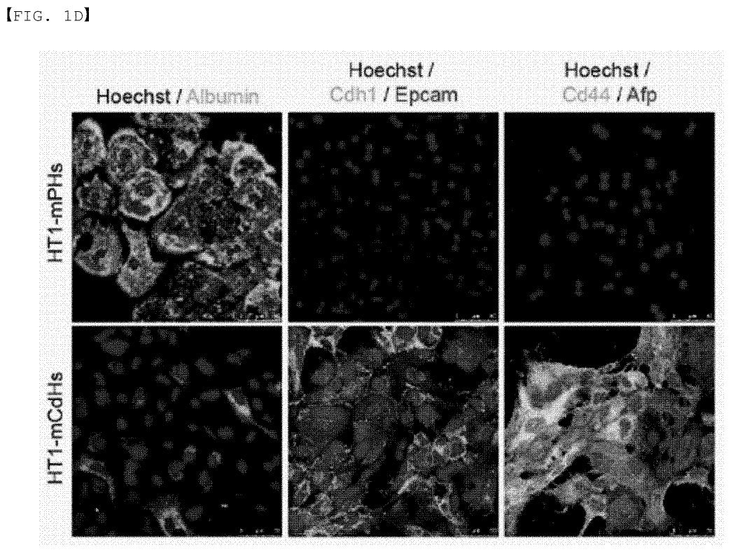

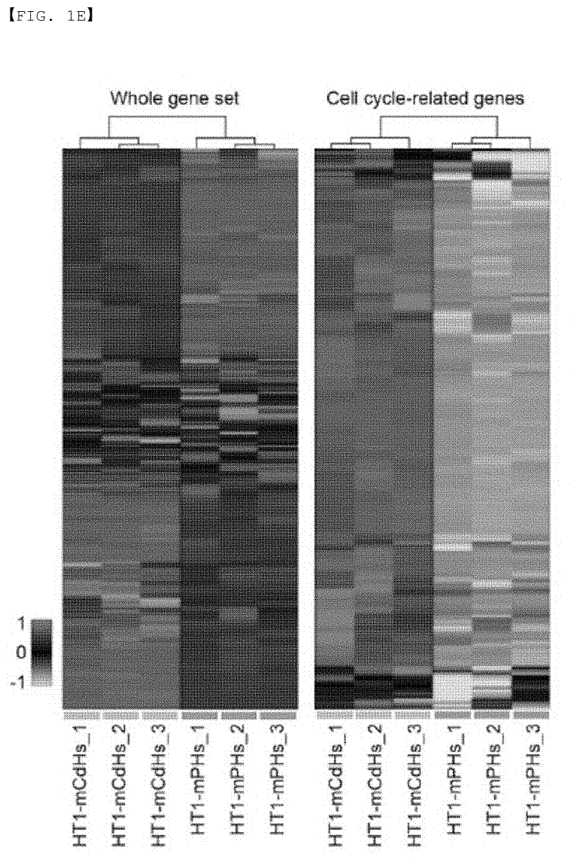

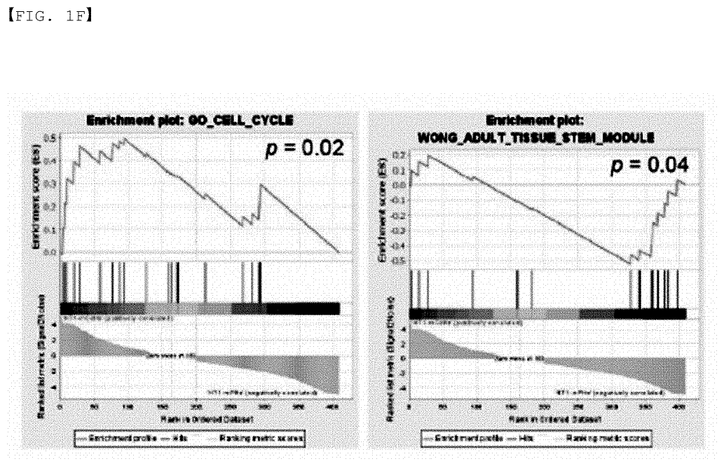

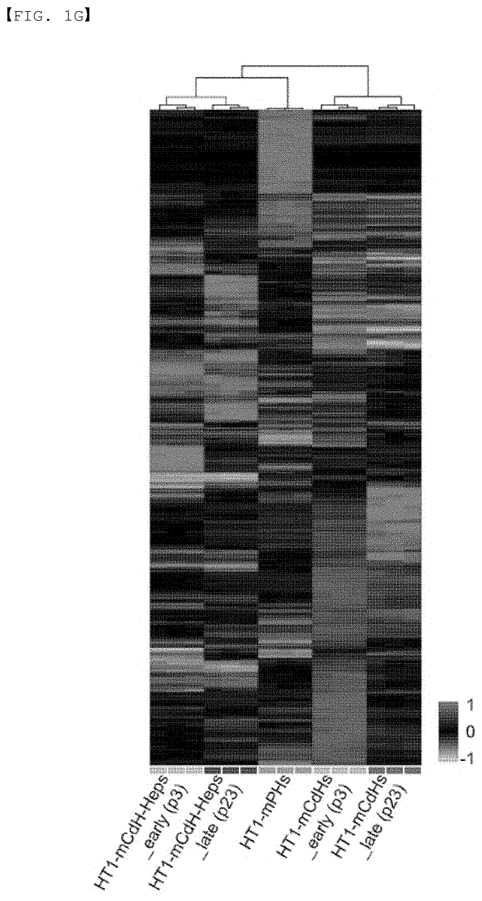

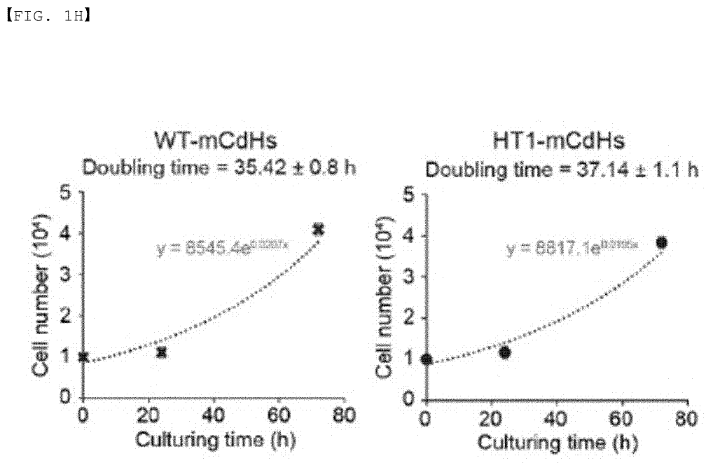

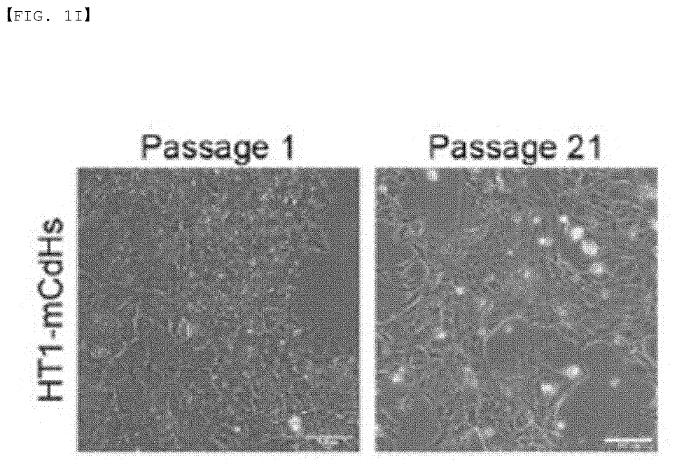

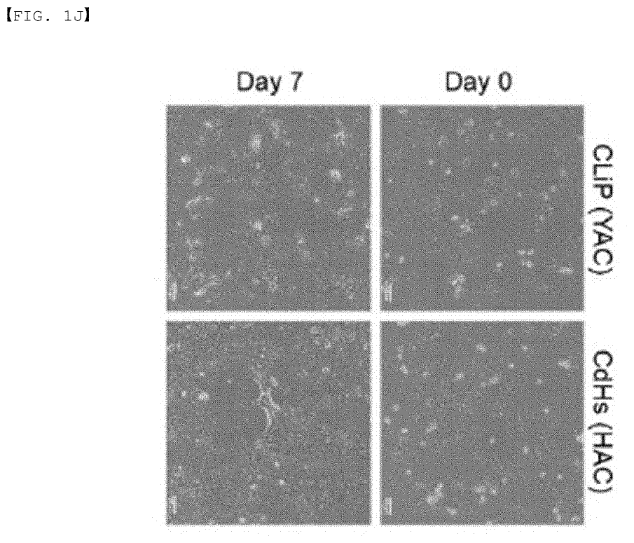

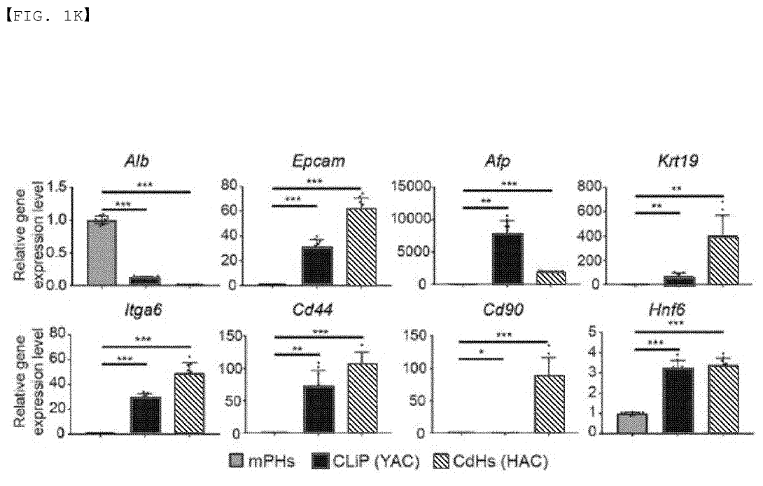

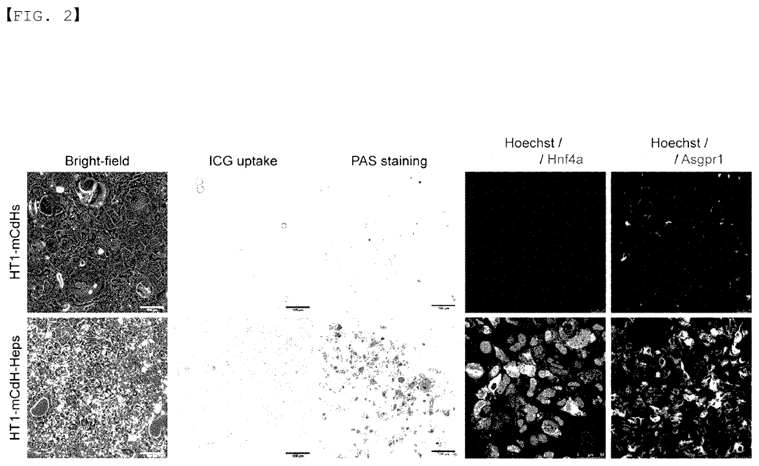

To generate mCdHs from a HT1 model mouse, the present inventors investigated whether the previous protocols used for human hepatocyte reprogramming could also be applied to mouse-derived PHs (HT1-mPHs). To this end, the present inventors treated the PHs with one growth factor and two compounds (also referred to as HAC), which are a hepatic growth factor (HGF), A83-01 (TGF-β inhibitor) and CHIR99021 (GSK-3 inhibitor) (FIG. 1A). As a result, as shown in FIG. 1B, it was confirmed that HAC-treated HT1-mPHs exhibited the morphology of small epithelial cells three days after treatment and that the cell population was expanded to cover plates after 8 days. In addition, as shown in FIGS. 1C and 1D, it was confirmed that the cells expressed hepatic stem cell-specific markers including Krt19, Sox9 and Afp, and the present inventors confirmed that these cells are chemically-derived hepatic progenitor cells from a HT1 mouse (hereinafter, HT1-mCdHs).

To more specifically confirm the characteristics of HT1-mCdHs, RNA sequencing was performed. After hierarchical clustering analysis, as shown in FIG. 1E, it was confirmed that the whole gene expression pattern of HT1-mCdHs was different from that of HT1 mouse primary hepatocytes (HT1-mPHs), and particularly, the expression patterns of cell cycle-related genes highly expressed in HT1-mCdHs appeared differently. Such a result, as shown in FIG. 1F, showed that, even when gene set enrichment analysis (GSEA) was performed, a similar result is shown. However, as shown in FIGS. 1C and 1G, compared with chemically-derived hepatic progenitor cells from wild-type C57BL/6N mice (WTmCdHs), HT1-mCdHs did not show differences in gene expression level and proliferation capacity. Even when HT1-mCdHs were subcultured 23 times, the expression of the entire transcriptome was maintained, indicating that these cells are stable enough to be able to generate gene-corrected clones (FIG. 1I). When HT1-mCdHs were compared with CliPs prepared in Example 1-2, as shown in FIGS. 1J and 1K, it was confirmed that a similar level of gene expression is shown.

2-2. Confirmation of Bipotent Differentiation Capacity of HT1-mCdHs

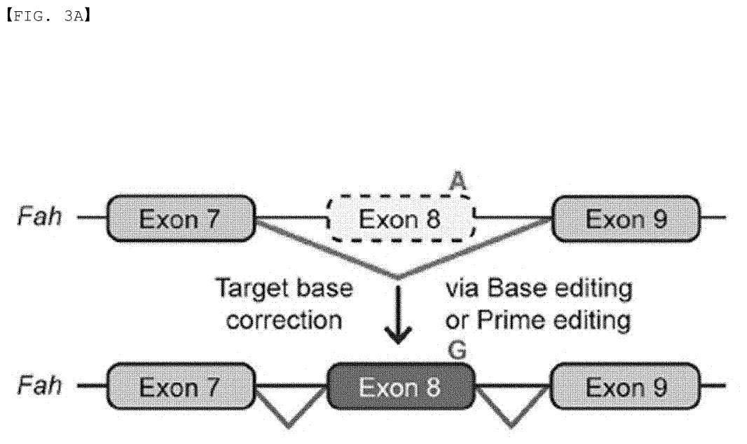

HT1 mouse-derived hepatic progenitor cells (HT1-mCdHs) have the capacity to differentiate into both mature hepatocytes and cholangiocytes. To confirm the differentiation capacity of HT1-mCdHs, the present inventors first cultured HT1-mCdHs under the condition of hepatic differentiation. The present inventors confirmed that, as shown in FIG. 2 showing the analyses of Indocyanine green (ICG) uptake and periodic acid-Schiff (PAS) staining, hepatocyte-like cells (HT1-mCdHs-Heps) differentiated from HT1-mCdHs acquired both mature hepatocyte morphology and mature liver characteristics. From the result of immunofluorescence, it was also shown that, in HT1-mCdHs-Heps, mature hepatocytes-specific markers, including albumin, Hnf4a, Krt18, and Asgpr1, were expressed after hepatic differentiation. Such a result shows that mCdHs can re-differentiate into mature hepatocytes under proper conditions.

To confirm whether the HT1-mCdHs of the present invention can differentiate into cholangiocytes, other than hepatocytes, an additional experiment including a 3D culturing method was performed. Specifically, it was confirmed that the cells (HT1-mCdH-Chols) differentiated in this way form a characteristic tubular-like structure, and compared with HT1-mCdHs, express cholangiocyte-specific markers such as Krt19, Cftr, Ae2, and Aqpr1 at higher levels.

From the above results, the present inventors specifically confirmed that chemically-derived hepatic progenitor cells having bipotent differentiation capacity were established by chemical treatment of primary hepatocytes isolated from the HT1 cells.

### Example 3. Adenine Base Editing and Prime Editing for Fah Mutation Correction

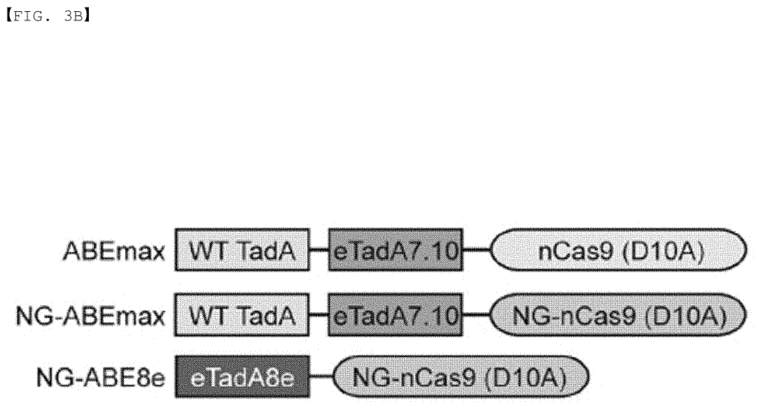

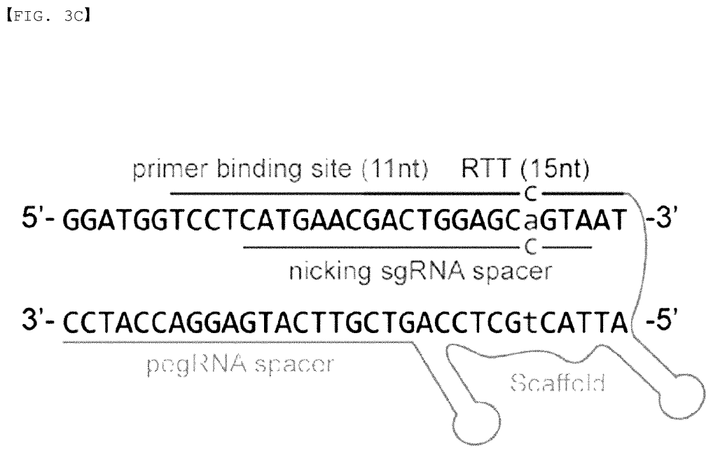

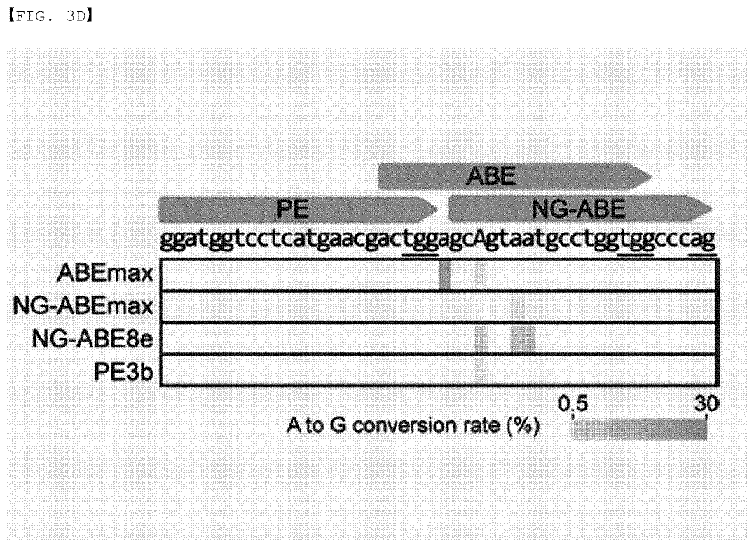

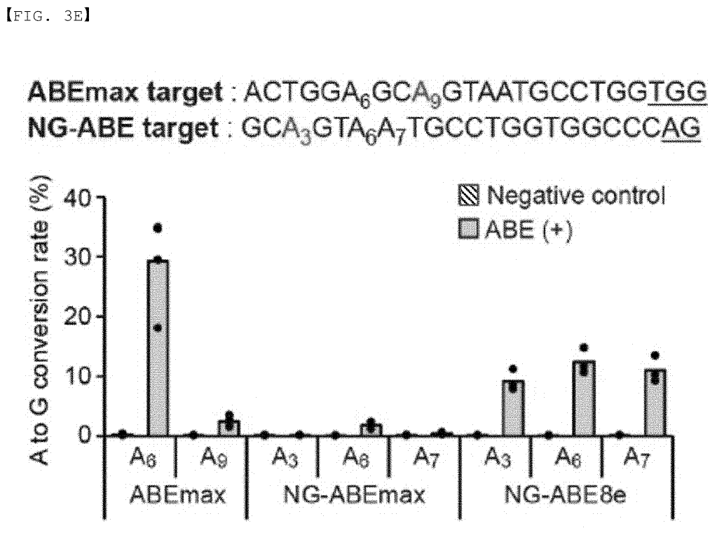

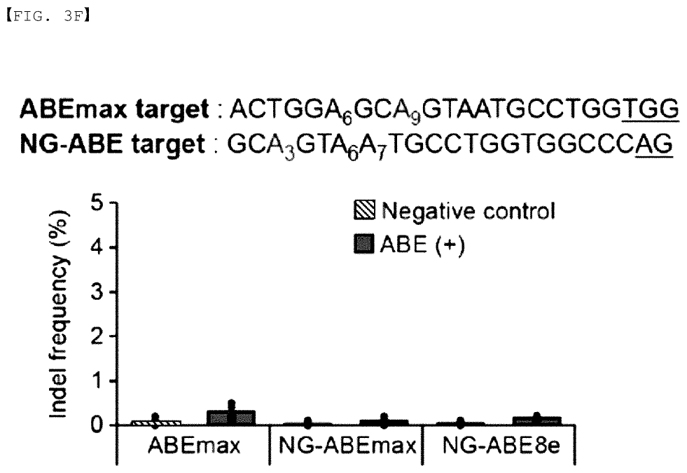

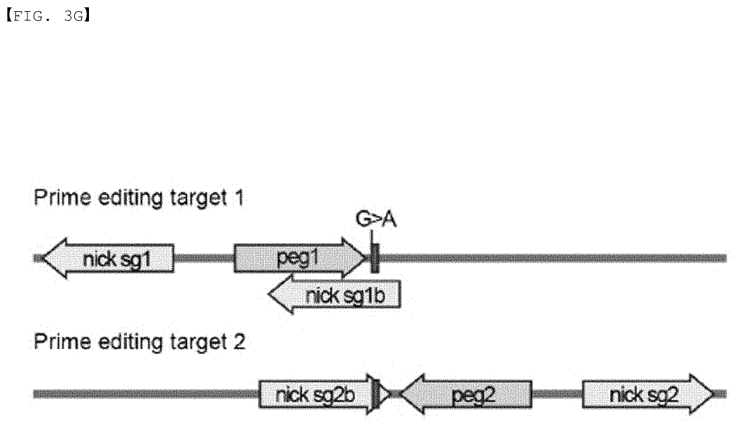

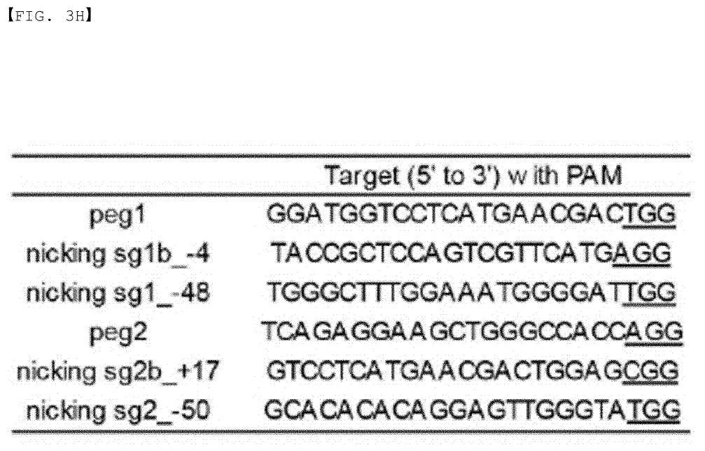

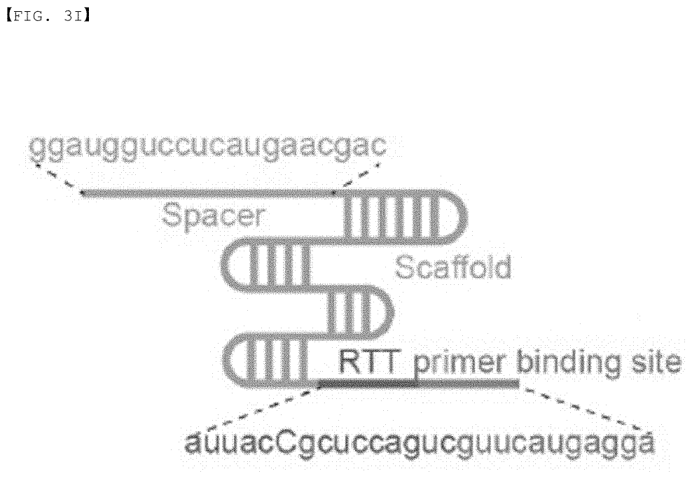

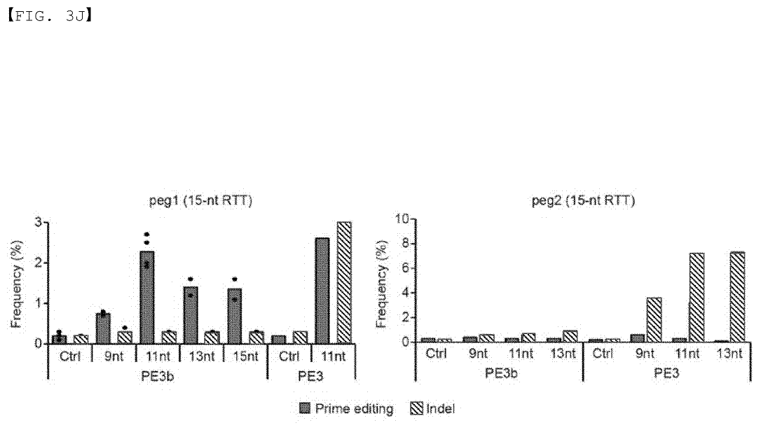

The Fah mutation present in a HT1 model mouse refers to the generation of a non-functional Fah enzyme by skipping exon 8 during splicing due to a G>A mutation at the 3′end of exon 8 (FIG. 3A). To correct the mutation, both ABE (FIG. 3B) and PE (FIG. 3C) were tested. First, single guide RNA (sgRNA) was designed for use with previously developed ABE and ABEmax, recognizing an NGG protospacer adjacent motif. When using this method, the point mutation was positioned near, but not in the editing window (positions 4 to 7). Electroporation was used to transfect an ABEmax-encoding plasmid, together with a sgRNA-encoding plasmid, into HT1-mCdHs, and 3 days later, a bulk cell population was subjected to high-throughput sequencing, showing that the adenosine (A9) at the position where a change was required was base-converted at a level of 2.4% on average, whereas bystander A (A6) was base-converted at a level of 29.3%. This is an expected result because the ABEmax is known to more easily edit adenosine at position 6 compared to position 9.

To increase the target adenosine conversion rate, ABE variants disclosed in Richter et al. (Nat. Biotechnol. 38, 883-891.), recognizing recently developed NG PAM, were applied. As a result, as shown in FIGS. 3B, 3D, 3E and 3F, the adenosine conversion rate of the adenosine (now A3) at a target position is 9.2% on average, indicating a significant improvement over the conventional conversion rate of 2.4%.

Finally, to precisely convert only a target base without the conversion of a bystander base, prime editors (PEs) were tested. A prime editor system (PE3 or PE3b) needs additional nicking sgRNA and prime editing guide RNA (pegRNA), and the pegRNA consists of a guide RNA spacer sequence, a reverse transcription template (RTT) and a primer-binding site (FIG. 3C). To optimize the editing activity of PE3, two different PE targets were designed, various pegRNAs including a 15-nt RTT coupled to primer-binding sites with different lengths ranging from 9 to 15 nt were tested (FIGS. 3G and 3J), and two nicking sgRNAs were designed for each pegRNA to utilize both PE3 and PE3b. As a result, through the test, pegRNA1 with an 11-nt prime-binding site and nicking sgRNA1b were selected, and through this, the highest conversion rate (2.3% on average) was obtained without bystander base conversion (FIGS. 3D and 3J).

Through the above results, it was confirmed that the present inventors established ABE- and PE-based mutation correction strategies for optimal mutant gene correction in HT1-mCdHs.

### Example 4. Confirmation of Off-Target Effect of Gene Editing Technology

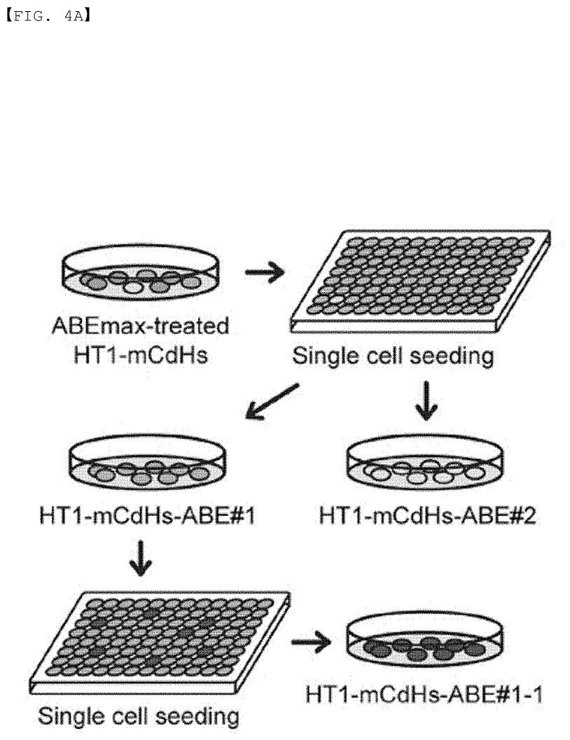

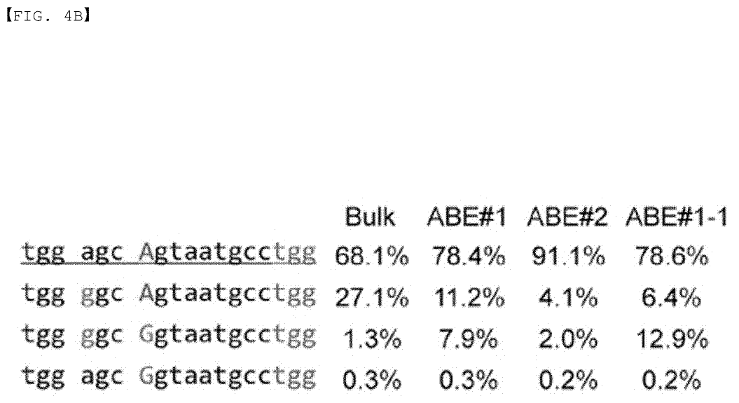

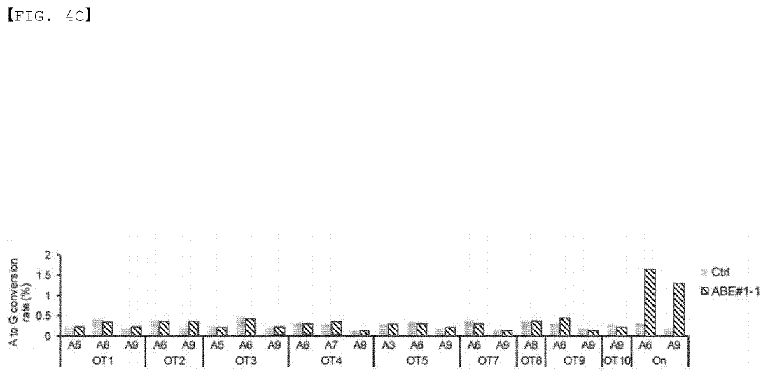

To check whether an off-target effect occurs when using the gene editing technology of the present invention, experiments were performed on ABEmax-treated HT1-mCdHs. Specifically, to isolate a clonal cell line including a corrected Fah gene, ABE-treated bulk cells were diluted, and cell lines including a corrected Fah gene were selected by performing high-throughput sequencing. As a result, as shown in FIG. 4A, two cell lines showing high correction rates, HT1-mCdHs-ABE #1 and HT-mCdHs-ABE #2, were selected. It was confirmed that, as shown in FIG. 4B, these cell lines are associated with at least four different sequence patterns, and to exclude the possibility that these cell lines do not consist of identical clones, the HT1-mCdHs-ABE #1 cell line was re-diluted to separate single cells, and the cells were subjected to high-throughput sequencing to reconfirm the presence of a corrected gene in each cell line. It was observed that all of the obtained clones had at least four different sequence patterns, suggesting that HT1-mCdHs might be polyploid, similar to primary hepatocytes, because it is already known from previous studies that hepatocytes in adult mammals have polyploid characteristics and about 90% of the total rodent hepatocyte population is polyploid. When these diploid HT1-mCdHs were isolated and cultured for 14 days, it was confirmed that the polyploid distribution thereof shifted to tetraploid or octaploid as in the original populations.

Among the sets of the HT1-mCdHs-ABE #1 clones, a cell line showing the highest correction frequency (13.1%) of a target sequence was selected and referred to as HT1-mCdHs-ABE #1-1. To examine an ABEmax-mediated off-target effect in HT1-mCdHs-ABE #1-1, as in Example 1-12, restriction enzyme V (endonuclease V, EndoV)-coupled Digenome-seq was performed. As a result, from the cells, 11 ex vivo cleavage sites, including a target site were confirmed, and 10 potential off-target sites were identified in silico, using Cas-OFFinder software. These results are shown in Table 2 below.

TABLE 2
Potential off-target sites captured by Digenome-seq

| | OT | Chr: position | Cleavage score |
|---|---|---|---|
| Control genomic | — | chr19: 60315442 | 6.37 |
| DNA | — | chr8: 72112588 | 5.87 |
| | — | chr3: 28912995 | 5.46 |
| | — | chr9: 37000968 | 4.38 |
| | — | chr18: 60089364 | 4.26 |
| | — | chr9: 104123406 | 4.06 |
| | — | chr19: 60141777 | 4.01 |
| | — | chr8: 75136581 | 4 |
| E/Endo V-treated | 1 | chr3: 117504849 | 6.01 |
| genomic DNA | 2 | chr3: 84545503 | 5.5 |
| | 3 | chr9: 74162358 | 5.45 |
| | 4 | chr17: 45840609 | 5.24 |
| | 5 | chr19: 37449202 | 5.02 |
| | 6 | chr19: 8955071 | 4.65 |
| | 10 | chr19: 60680279 | 4.62 |
| | On | chr7: 84595450 | 4.48 |
| | 7 | chr3: 152059170 | 4.16 |
| | 8 | chr7: 16025764 | 4.12 |
| | 9 | chr17: 47517533 | 4.1 |

Genomic sites predicted using Cas-OFFinder

| | Target (5′ to 3′) with | | | |
|---|---|---|---|---|
| OT | PAM sequence | chr: position | Direction | Mismatches |
| 1 | ACTGaAGCAGTAAaGCCTGGTGG | chr12: 25231004 | − | 2 |
| 2 | AaTGGAGCAGTAATGgCaGGAGG | chr7: 16834673 | + | 3 |
| 3 | tCTGaAGCAGTAgTGCCTGGGGG | chr4: 76815045 | − | 3 |
| 4 | ACTGGAaCAGTAgTGCtTGGGGG | chr5: 25858421 | − | 3 |
| 5 | ACaGGAGCAGgAAgGCCTGGGGG | chr16: 90485210 | − | 3 |
| 6 | ACTGGAGCAGTAggGCaTGGGGG | chr19: 46263600 | − | 3 |
| 7 | gCTGGAGCAaTAATGgCTGGTGG | chr17: 70886653 | − | 3 |
| 8 | ACTGGtGatGTAATGCCTGGGGG | chr10: 86492242 | − | 3 |
| 9 | ACTGGAGCAGTAAgGCCaGaGGG | chr18: 5622427 | + | 3 |
| 10 | gCTGGtGCAGTgATGCCTGGAGG | chr18: 25501316 | + | 3 |

When high-throughput sequencing was performed on each cleavage site of HT1-mCdHs-ABE #1-1, as shown in FIG. 4C, no significant level of off-target effect was identified.

### Example 5. Confirmation of Therapeutic Effect of HT1-mCdHs with Ex Vivo Gene Correction by ABE on Tyrosinemia Type I

5-1. Confirmation of Therapeutic Effect of Cells Whose Gene has been Corrected by ABE

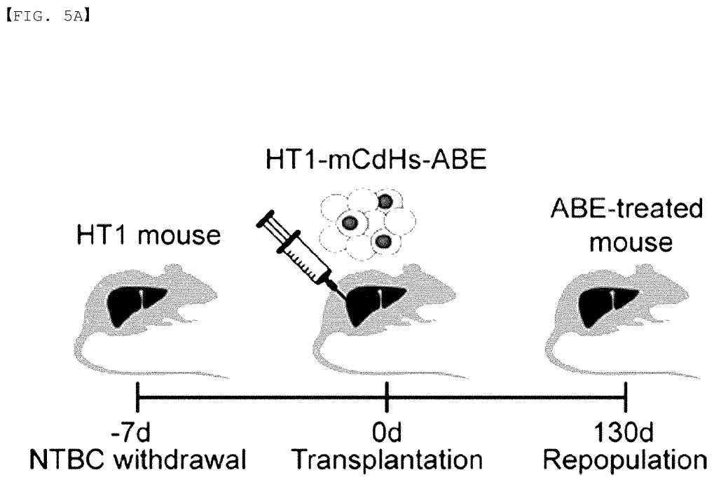

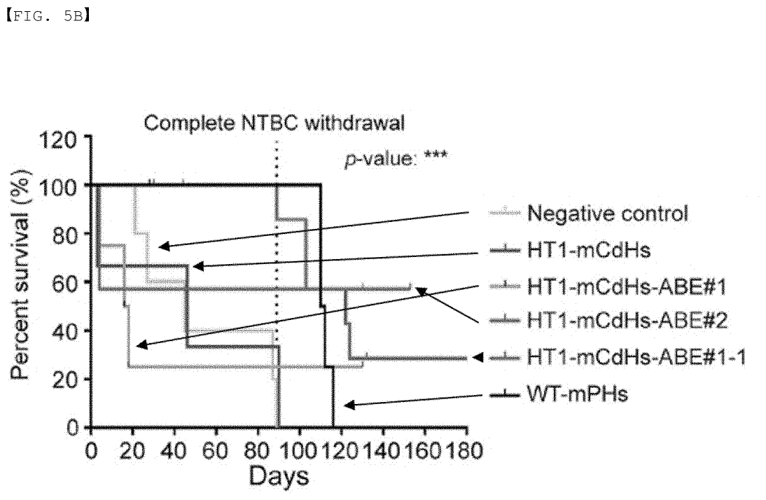

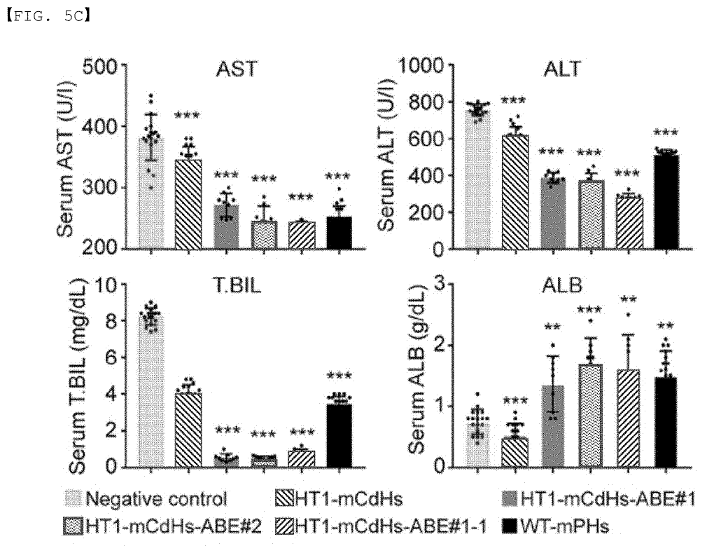

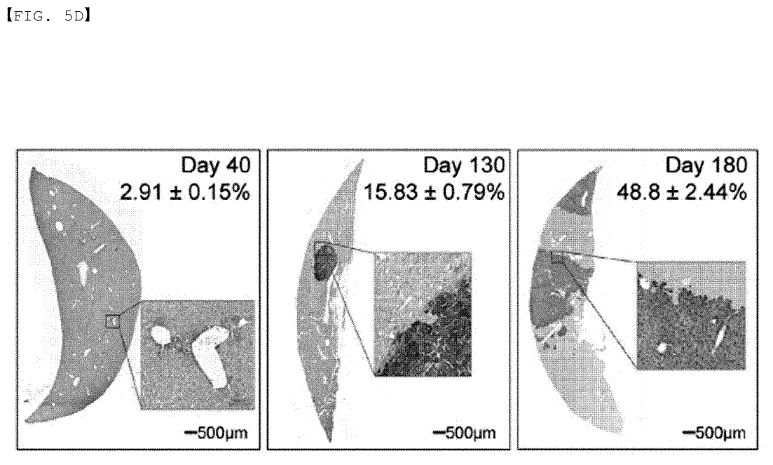

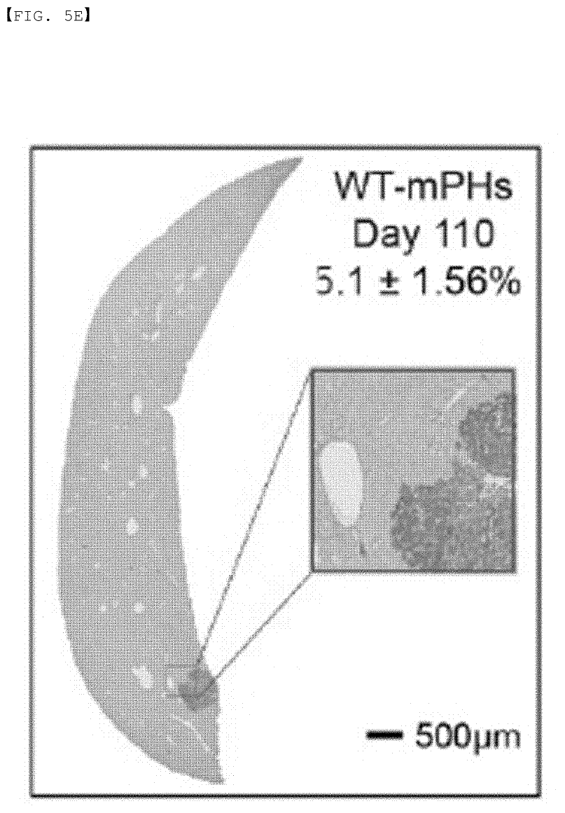

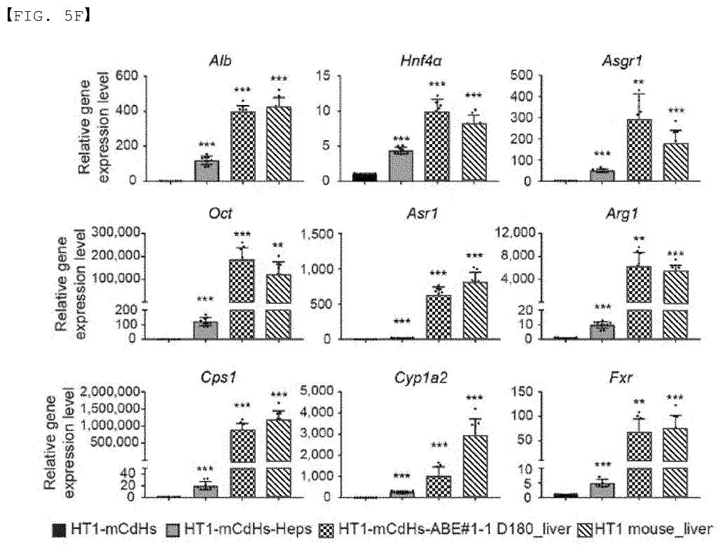

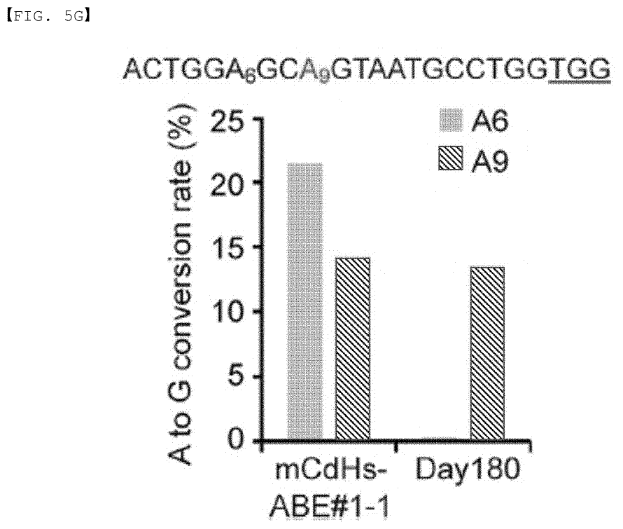

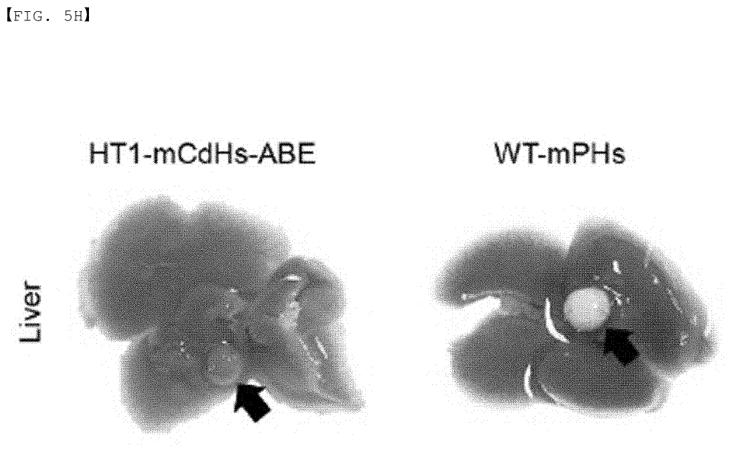

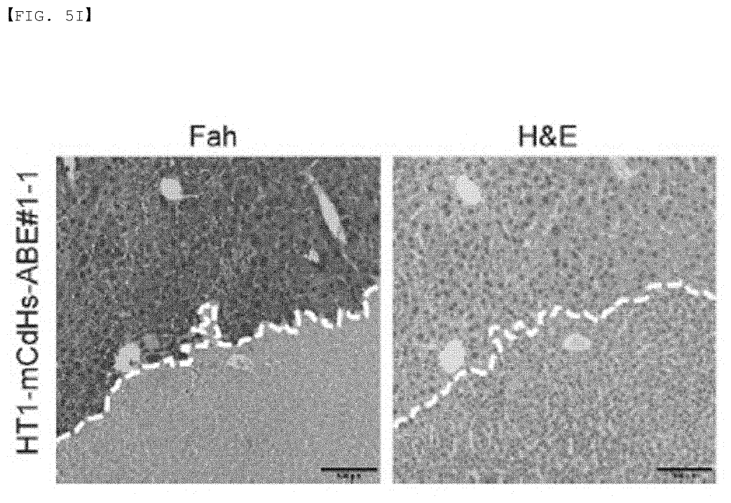

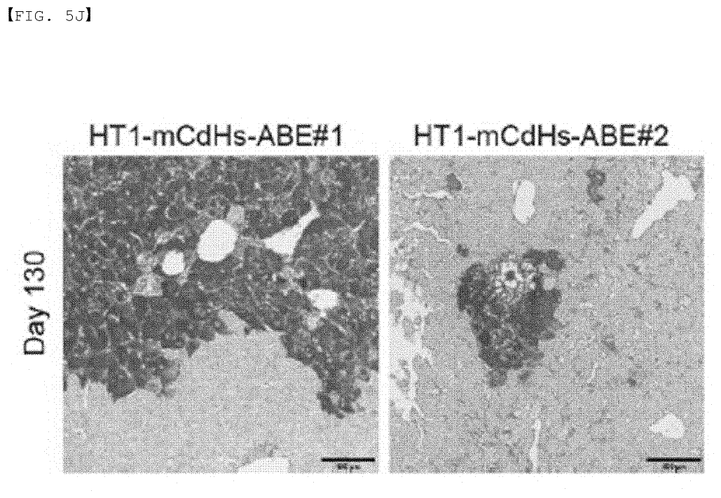

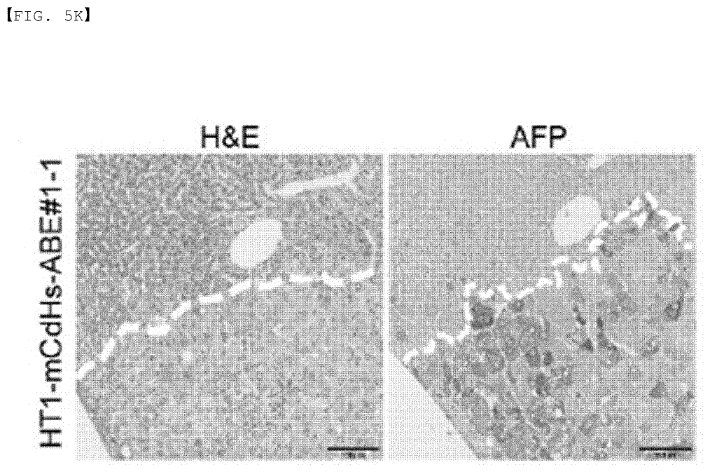

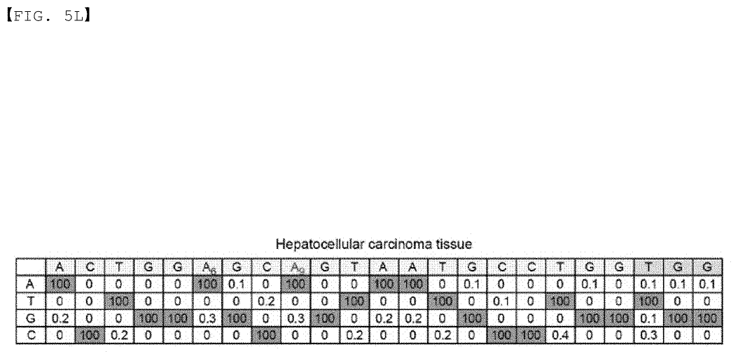

The present inventors tested whether the corrected mCdHs could exhibit a reliable repopulation capacity in HT1 mice, and thus exhibit therapeutic potential. The present inventors transplanted the partially corrected HT1-mCdHs-ABE #1-1 cell line, in which a significant off-target effect was not identified, into the spleen of a HT1 mouse. Specifically, seven days before transplantation, NTBC was withdrawn from drinking water of nine HT1 mice to induce liver damage, resulting in easy transplantation of HT1-mCdHs-ABE #1-1 (FIG. 5A). Phosphate-buffered saline (PBS) and HT1-mCdHs were used as negative controls, and a primary hepatocyte-transplanted group derived from a wild-type mouse (WT-mPHs) was used as a positive control. After transplantation, the mice in the PBS-injected group (5 mice) and HT1-mCdHs-transplanted group (5 mice) began to rapidly die, and as shown in FIG. 5B, all mice died by day 90, and it was also confirmed that all animals (5 mice) in the WT-mPHs-transplanted group were also dead by about day 120. However, two from the HT1-mCdHs-ABE #1-1-transplanted group (9 mice), which had been corrected ex vivo using the gene editing technology of the present invention, survived over 180 days. In the case of the two mice surviving more than 180 days, levels of serum biomarkers including aspartate transaminase (AST), alanine transaminase (ALT), total bilirubin, and albumin (ALB) showed that liver damage was significantly decreased after HT1-mCdHs-ABE #1-1 transplantation (FIG. 5C).

To confirm the repopulation capacity of HT1-mCdHs-ABE #1-1, the present inventors examined Fah-positive cell populations in the mice of the HT1-mCdHs-ABE #1-1-transplanted group at day 40, 130 and 180. It was confirmed that the Fah-positive cell populations were engrafted around the hepatic vein at day 40 after transplantation (FIG. 5D). After 130 days, the area colonized by the Fah-positive cells increased up to 15% of the liver section, and further increased almost up to 50% at day 180. These cells showed different morphology from the early primary hepatocytes (FIG. 5D). On the other hand, in the case of the primary hepatocyte-transplanted group, as shown in FIG. 5E, only about 5.1% Fah-positive cells were observed. In addition, when the HT1-mCdHs-ABE #1-1 of the present invention were transplanted, their therapeutic effect was identified, and then they were reisolated to confirm mRNA expression, as shown in FIG. 5F, the gene expression level was similar to that of the liver in the HT1 mouse, reconfirming the in vivo differentiation capacity of the cells of the present invention. From this result, it was confirmed that the ex vivo gene corrected HT1-mCdHs-ABE #1-1 of the present invention showed a significant target disease therapeutic effect, compared to the case of transplanting primary hepatocytes.

In addition, it was confirmed that the frequency of alleles in which only a target adenosine (A9) had been edited without editing of bystander adenosine abruptly increased (from 0.2% to 13.3%) at day 180, whereas the frequency of alleles in which both the target adenosine (A9) and bystander adenosine (A6) decreased in the HT1-mCdHs-ABE #1-1-transplanted mice (FIG. 5G). This indicates that when transplanted, cells containing the corrected alleles became dominant in the liver during cell replication, and the A6-transplanted cells were eliminated in vivo.

To investigate the reproducibility of the ex vivo gene editing strategy of the present inventors, the present inventors repeated experiments using other corrected mCdHs cell lines, such as HT1-mCdHs-ABE #1 and HT1-mCdHs-ABE #2 (FIG. 4B). The present inventors confirmed that the mice in the HT1-mCdHs-ABE #1-transplanted group (4 mice) and HT1-mCdHs-ABE #2-transplanted group (7 mice) survived for more than 130 days even when NTBC was not treated (FIG. 5B). In addition, levels of markers indicting liver damage were reduced (FIG. 5C). Likewise, although FAH-positive cell populations in these two groups were observed to show patterns similar to those in the HT1-mCdHs-ABE #1-1-transplanted group (FIGS. 5I to 5L), the frequency of the mutation-corrected sequence is lower than that of the HT1-mCdHs-ABE #1-1 group.

5-2. Confirmation of Stability of Cells Whose Gene has been Corrected by ABE

During the transplantation experiment, the present inventors confirmed that two of the 9 HT1-mCdHs-ABE #1-1-transplanted mice and 1 of the 5 WT-mPHs-transplanted mice had hepatocellular carcinoma (HCC). To confirm whether HCC was caused by HT1-mCdHs-ABE #1-1, sequencing was performed on cells of the HCC section, and as shown in FIGS. 5H to 5L, a gene corrected by the editing technology of the present invention could not be identified in the cells of the HCC section, confirming that HCC naturally occurs in the HT1 model mice in an NTBS-free environment as previously disclosed in Buitrago-Molina et al. (Hepatology Volume 58, Issue 3 p. 1143-1152), and cells corrected ex vivo by the editing technology of the present invention did not cause cancer.

### Example 6. Confirmation of Tyrosinemia Type I Therapeutic Effect of Cells Whose Gene has been Corrected Ex Vivo by PE

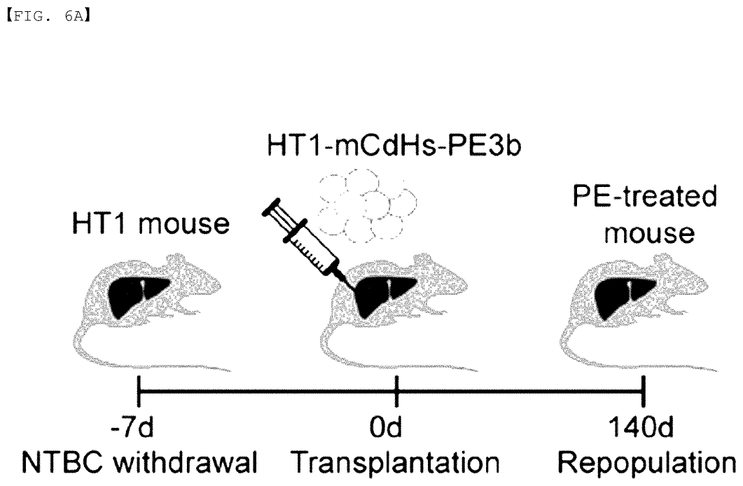

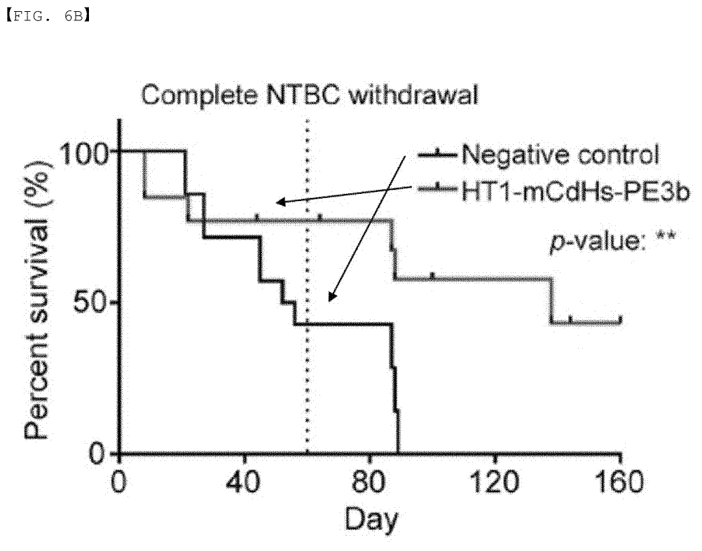

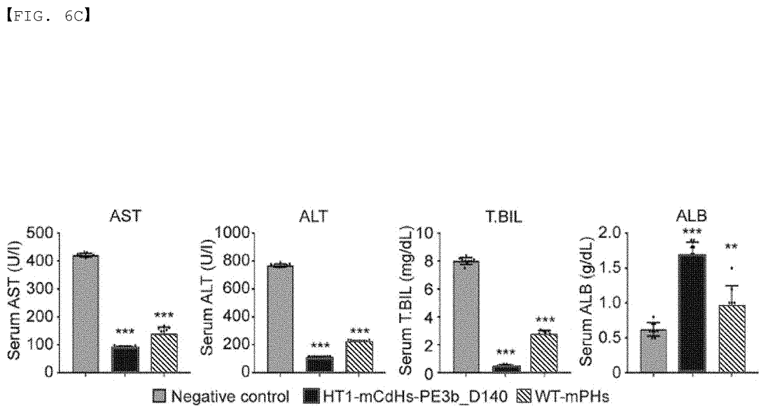

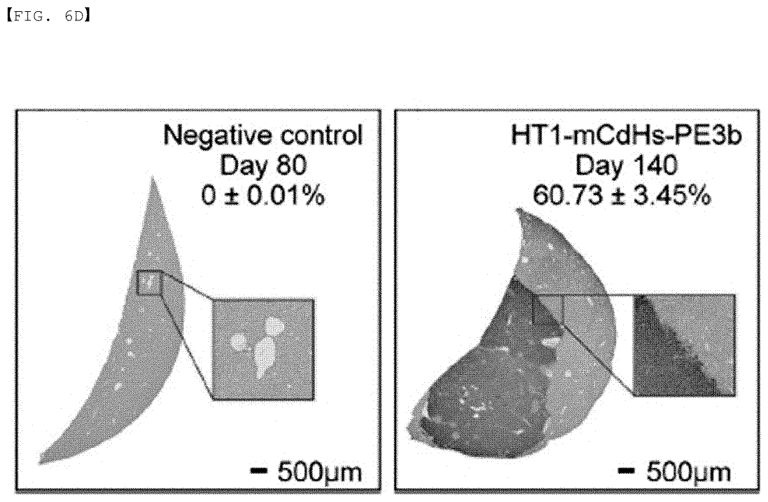

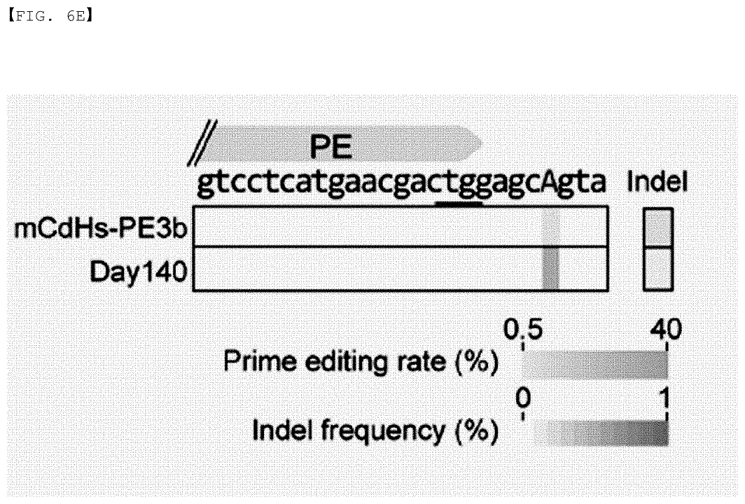

To confirm whether cells corrected by prime editing (PE), other than cells corrected by ABE, also exhibit a therapeutic effect on a disease caused by a gene mutation, experiments were performed using HT1-mCdHs-PE3b cells corrected by PE3b-mediated prime editing. In this case, unlike the ABE-mediated editing, a sufficient editing efficiency (2.3% on average) was shown and almost no bystander base correction was shown, using a bulk cell population, rather than an isolated clonal cell line. Specifically, similar to Example 5-2, as shown in FIG. 6A, NTBC was withdrawn from drinking water to induce liver damage and completely withdrawn by day 60. PBS-injected mice were used as a negative control.

The PBS-administered mice (9 mice), utilized as the control, rapidly died before day 90. However, 7 of the group of mice (13 mice) into which HT1-mCdHs-PE3b whose gene had been corrected by PE were transplanted survived for more than 160 days, indicating that the chemically-derived hepatic progenitor cells of the present invention, which had been subjected to gene correction using prime editing technology, might significantly treat HT1 disease even without NTBC (FIG. 6B). In the case of mice surviving more than 140 days of the mice, as shown in FIG. 6C, the expression of AST, ALT, T.BIL and ALB biomarkers in serum was significantly decreased, confirming the recovery of liver damage. In the case of the mice surviving more than 140 days, after immunohistochemistry, as shown in FIG. 6D, a Fah-positive cell population was observed, and cell proliferation was confirmed. In the case of the PBS-injected control, it was confirmed that there was no Fah-positive cell population. In addition, similar to the HT1-mCdHs-ABE #1-1-transplanted mice, in the liver of the HT1-mCdHs-PE3b-transplanted mice, as shown in FIG. 6E, an increasing frequency of the edited nucleotides was confirmed.

Through the above results, the present inventors showed that the ex vivo gene editing strategy is a reliable and solid approach for the treatment of HT1 disease in mice.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

It was confirmed that, when a cell therapeutic agent including cells in which a mutant gene has been corrected according to the present invention is used, compared to the case of transplanting conventional primary hepatocytes, there were fewer side effects such as an off-target effect and tumorigenesis, and a significant level of therapeutic effect on tyrosinemia type I was shown, so it is expected to be effectively used for treatment of a disease caused by a genetic mutation, such as tyrosinemia type I.

1. A method of preparing mutant gene-corrected cells, comprising:
(a) isolating cells from a subject;
(b) treating the isolated cells with a compound to prepare chemically-derived progenitor cells; and
(c) correcting a mutant gene of the chemically-derived progenitor cells ex vivo.

2. The method of claim 1, wherein the correcting of a gene is to correct a gene by an adenine base editor or prime editing.

3. The method of claim 1, wherein the corrected gene is selected from the group consisting of fumarylacetoacetate hydrolase (Fah), ATPase copper transporting beta (ATP7B), Serpin family A member 1 (SERPINA1), ATP binding cassette subfamily B member 4 (ABCB4), aldolase, fructose-bisphosphate B (ALDOB), glycogen branching enzyme (GBE), Solute Carrier Family 25 Member 13 (SLC25A13), cystic fibrosis transmembrane conductance (CFTR), and ALMS1 Centrosome And Basal Body Associated Protein (ALMS1) genes.

4. The method of claim 1, wherein the isolated cells are primary hepatocytes.

5. The method of claim 1, wherein the compound used to treat the isolated cells is one or more selected from the group consisting of a hepatic growth factor, A83-01 and CHIR99021.

6. The method of claim 1, wherein the chemically-derived progenitor cells are chemically-derived hepatic progenitor cells.

7. A cell therapeutic agent, comprising the mutant gene-corrected cells prepared by the method of claim 1 or a cell population thereof as an active ingredient.

8. The cell therapeutic agent of claim 7, which is used to treat a disease caused by a gene mutation.

9. A pharmaceutical composition for preventing or treating a genetic mutation-related disease, comprising the cell therapeutic agent of claim 8.

10. The composition of claim 9, wherein the genetic mutation-related disease is selected from the group consisting of tyrosinemia type I, phenylketonuria, Wilson's disease, alpha-1 antitrypsin deficiency, progressive familial intrahepatic cholestasis type 3, hereditary fructose intolerance, glycogen storage disease type IV, argininosuccinate lyase deficiency, citrin deficiency, neonatal intrahepatic cholestasis by citrin deficiency, cholesteryl ester storage disease, cystic fibrosis, hereditary hemochromatosis, and Alström syndrome.

11. A method of preventing or treating a genetic mutation-related disease, comprising:
administering the pharmaceutical composition of claim 9 to a subject.

12. A use of the pharmaceutical composition of claim 9 for preventing or treating a genetic mutation-related disease.
